(19) United States
(12) Patent Application Publication
(10) Pub. No.: US 2024/0377746 A1
(43) Pub. Date: Nov. 14, 2024

(54) HARDMASK COMPOSITION, HARDMASK LAYER, AND METHOD OF FORMING PATTERNS

(71) Applicant: SAMSUNG SDI CO., LTD., Yongin-si (KR)

(72) Inventor: Seil CHOI, Suwon-si (KR)

(21) Appl. No.: 18/583,033

(22) Filed: Feb. 21, 2024

(30) Foreign Application Priority Data
May 8, 2023 (KR) 10-2023-0059327

(51) Int. Cl. G03F 7/11; C07C 215/68; C07C 215/74; C07C 225/22; C07D 333/22; C09D 165/00; H01L 21/308; H01L 21/32; H01L 21/3213

(52) U.S. Cl. CPC G03F 7/11; C07C 215/68; C07C 215/74; C07C 225/22; C07D 333/22; C09D 165/00; C07C 2603/50; H01L 21/3081; H01L 21/32; H01L 21/32139

(57) ABSTRACT

A hardmask composition, including a hardmask layer including a cured product of the hardmask composition, and a method of forming patterns using the hardmask layer including a cured product of the hardmask composition, the hardmask composition including a compound represented by Chemical Formula 1; and a solvent,

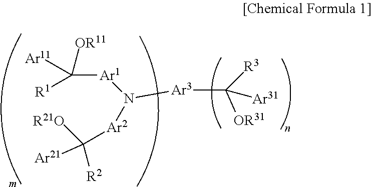

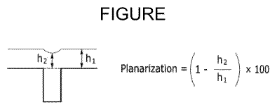

### CROSS-REFERENCE TO RELATED APPLICATION

This application claims priority to and the benefit of Korean Patent Application No. 10-2023-0059327 filed in the Korean Intellectual Property Office on May 8, 2023, the entire contents of which are incorporated herein by reference.

### BACKGROUND

### 1. Field

Embodiments relate to a hardmask composition, a hardmask layer, and a method of forming patterns.

### 2. Description of the Related Art

Recently, the semiconductor industry has developed to an ultra-fine technique having a pattern of several to several tens nanometer size. Such ultrafine technique may utilize lithographic techniques.

Some lithographic techniques may include providing a material layer on a semiconductor substrate; coating a photoresist layer thereon; exposing and developing the same to provide a photoresist pattern; and etching a material layer using the photoresist pattern as a mask.

According to small-sizing the pattern to be formed, it may be difficult to provide a fine pattern having an excellent profile by only using some lithographic techniques. Accordingly, an auxiliary layer, called a hardmask layer, may be formed between the material layer and the photoresist layer to provide a fine pattern.

### SUMMARY

The embodiments may be realized by providing a hardmask composition including a compound represented by Chemical Formula 1; and a solvent:

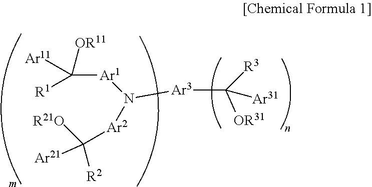

wherein, in Chemical Formula 1, Ar¹ to Ar³, Ar¹¹, Ar²¹, and Ar³¹ are each independently a substituted or unsubstituted C6 to C30 aromatic hydrocarbon group, a substituted or unsubstituted C3 to C30 hetero aromatic hydrocarbon group, a group in which two or more of the aromatic hydrocarbon groups, heteroaromatic hydrocarbon groups, or a combination thereof are linked by a single bond or a substituted or unsubstituted C1 to C10 alkylene group, or a combination thereof,
R¹ to R³ are each independently hydrogen, deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C1 to C30 saturated or unsaturated heteroaliphatic hydrocarbon group, a substituted or unsubstituted C3 to C30 saturated or unsaturated heteroalicyclic hydrocarbon group, a substituted or unsubstituted C6 to C30 aromatic hydrocarbon group, a substituted or unsubstituted C4 to C30 heteroaromatic hydrocarbon group, or a combination thereof,
R¹¹, R²¹, and R³¹ are each independently hydrogen, deuterium, a substituted or unsubstituted C1 to C30 saturated or unsaturated aliphatic hydrocarbon group, a substituted or unsubstituted C3 to C30 saturated or unsaturated alicyclic hydrocarbon group, or a substituted or unsubstituted C6 to C30 aromatic hydrocarbon group,
n is an integer of 0 to 2, and
m is an integer of 1 to 4.
Ar¹ to Ar³ in Chemical Formula 1 may each independently be a substituted or unsubstituted group of a moiety of Group 1:

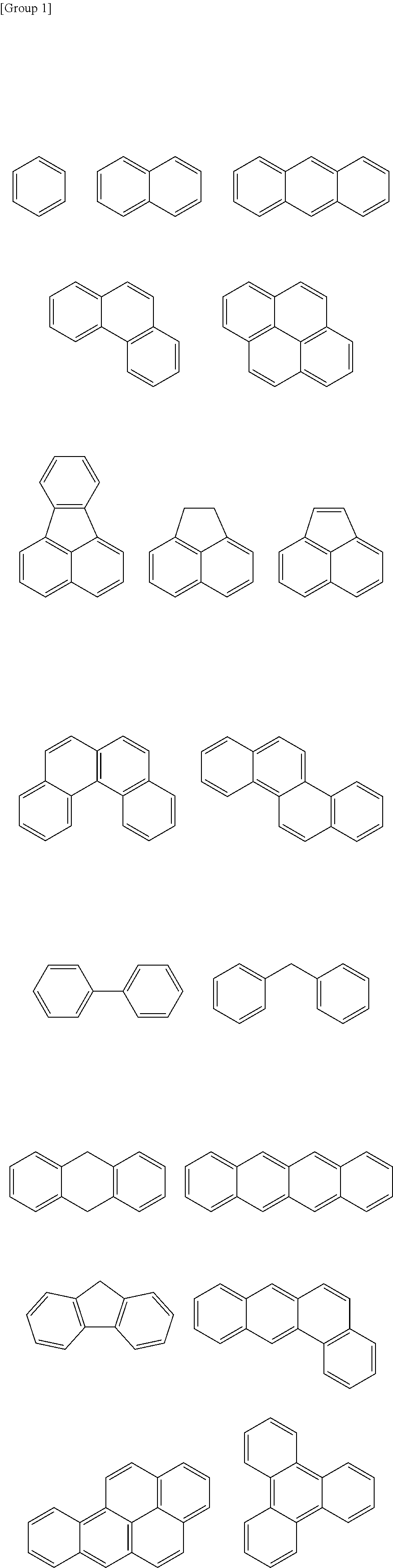

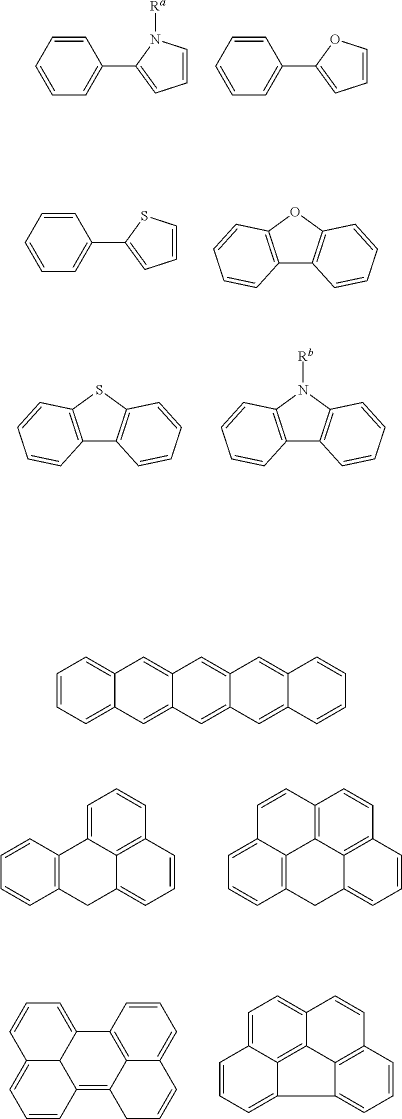

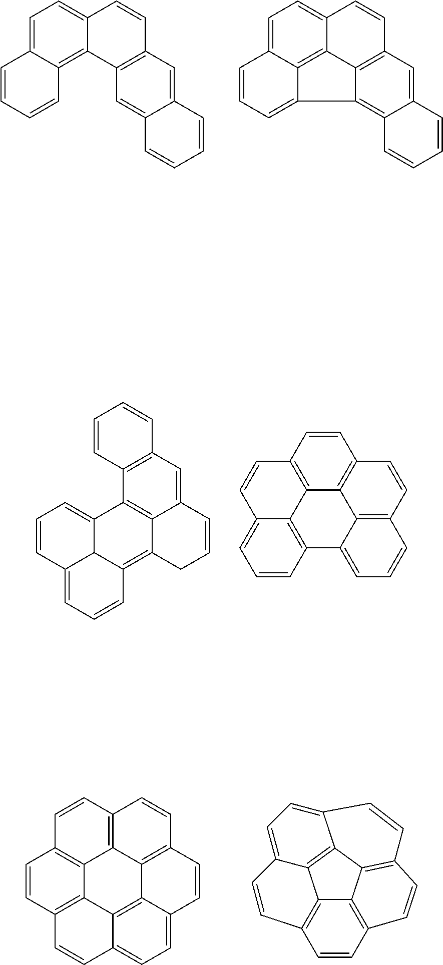

wherein, in Group 1, R^{a} and R^{b} may each independently be hydrogen, deuterium, a substituted or unsubstituted C1 to C10 alkyl group, or a substituted or unsubstituted C6 to C20 aryl group.
Ar¹ to Ar³ in Chemical Formula 1 may each independently be a substituted or unsubstituted group of a moiety of Group 1-1:

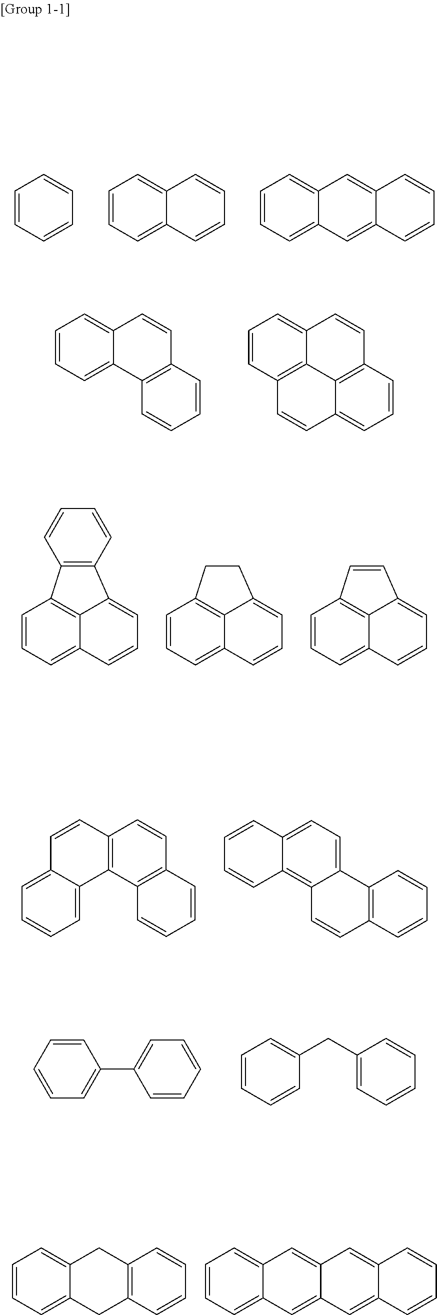

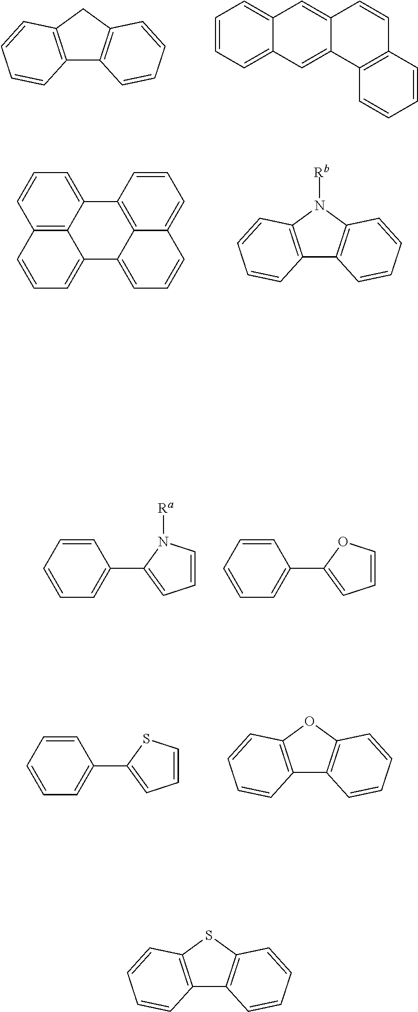

wherein, in Group 1-1, R^{a} and R^{b} may each independently be hydrogen, deuterium, a substituted or unsubstituted C1 to C10 alkyl group, or a substituted or unsubstituted C6 to C20 aryl group.
Ar¹¹, Ar²¹, and Ar³¹ in Chemical Formula 1 may each independently be a substituted or unsubstituted C6 to C30 aromatic hydrocarbon group.
Ar¹¹, Ar²¹, and Ar³¹ in Chemical Formula 1 may each independently be a substituted or unsubstituted group of a moiety of Group 2:

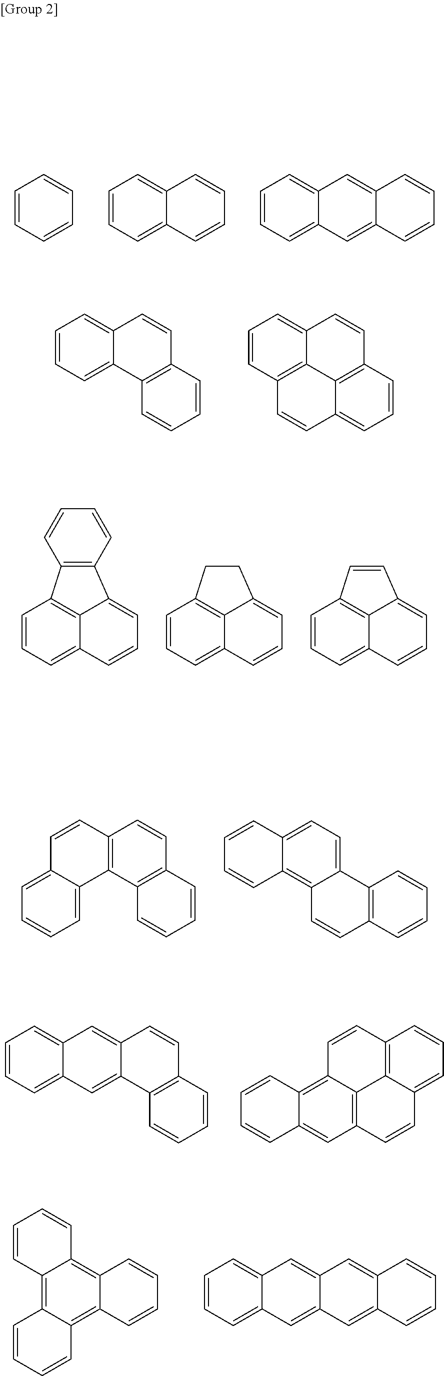

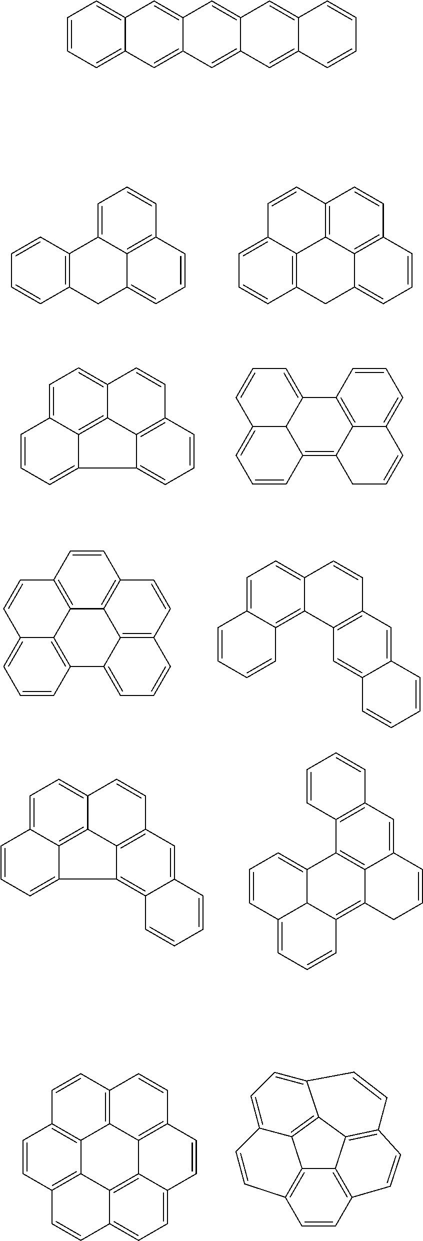

Ar¹ to Ar³ in Chemical Formula 1 may each independently be a substituted or unsubstituted group of a moiety of Group 1-2, and Ar¹¹, Ar²¹, and Ar³¹ may each independently be a substituted or unsubstituted group of a moiety of Group 2-1:

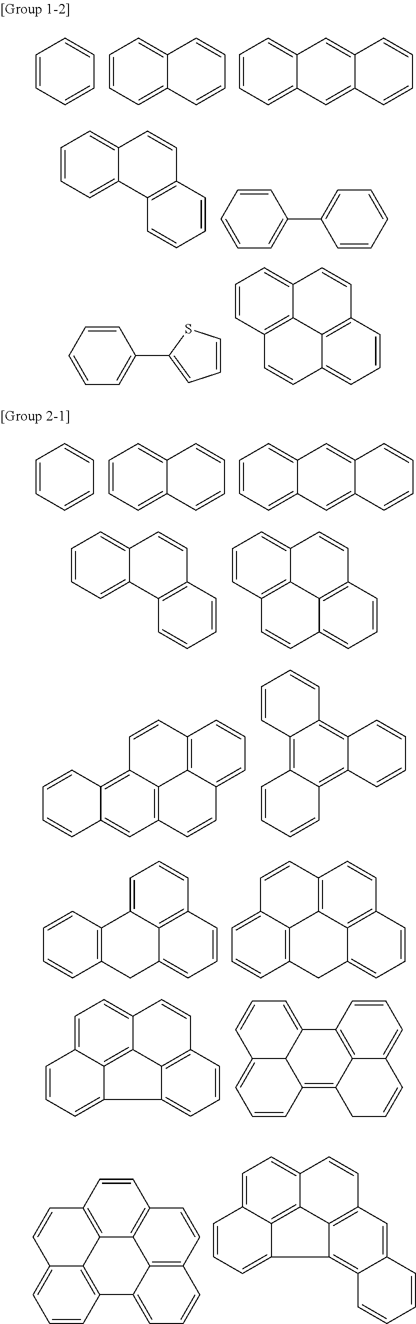

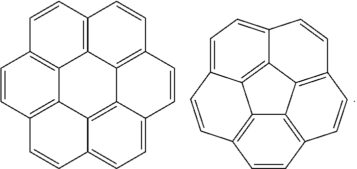

In Chemical Formula 1, R¹ to R³ may each independently be hydrogen, deuterium, a substituted or unsubstituted C1 to C30 saturated or unsaturated aliphatic hydrocarbon group, a substituted or unsubstituted C6 to C30 aromatic hydrocarbon group, or a combination thereof, and R¹¹, R²¹, and R³¹ may each independently be hydrogen, deuterium, or a substituted or unsubstituted C1 to C30 saturated or unsaturated aliphatic hydrocarbon group.

In Chemical Formula 1, R¹ to R³, R¹¹, R²¹, and R³¹ may each independently be hydrogen, deuterium, a substituted or unsubstituted C1 to C20 alkyl group, a substituted or unsubstituted C6 to C20 aromatic hydrocarbon group, or a combination thereof.

In Chemical Formula 1, Ar¹ and Ar², Ar¹¹ and Ar²¹, R¹ and R², or R¹¹ and R²¹ may be the same as each other.

The compound represented by Chemical Formula 1 may be represented by one of Chemical Formula 2 to Chemical Formula 15:

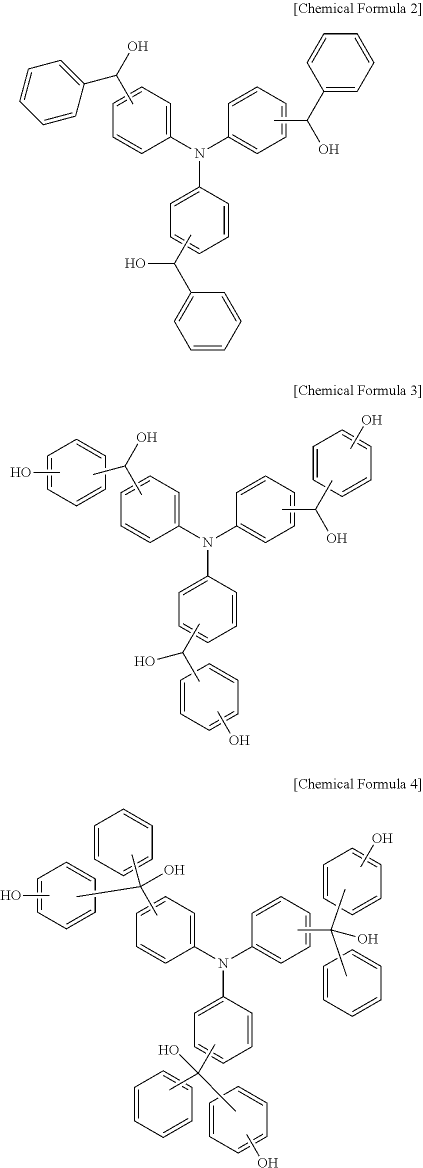

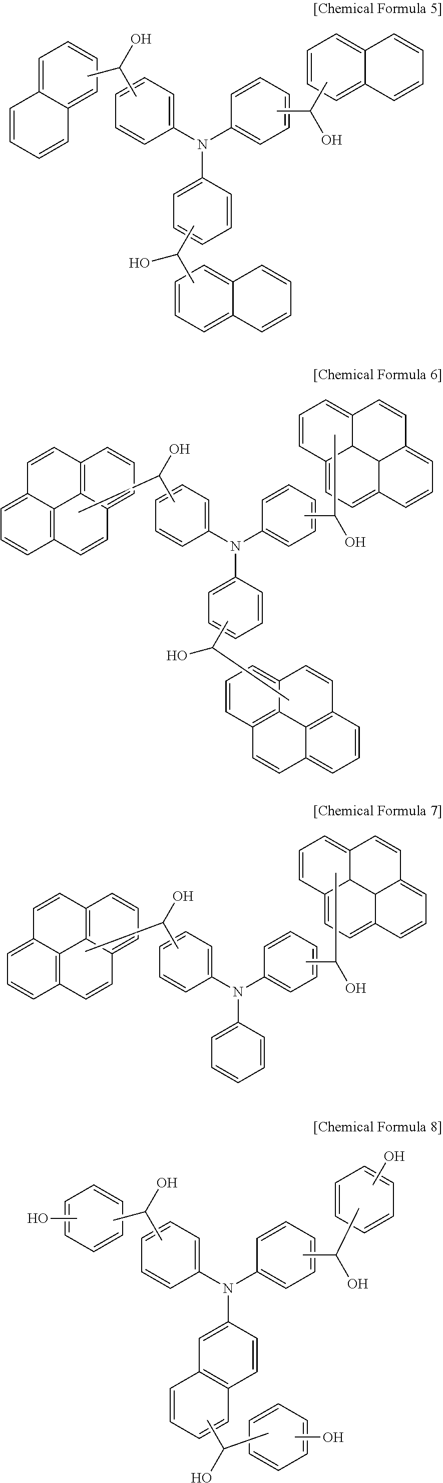

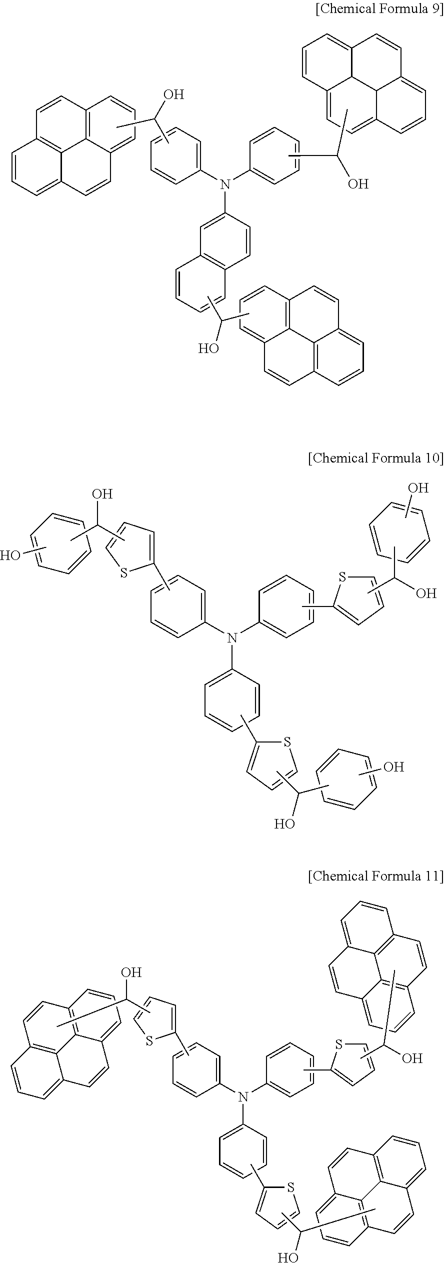

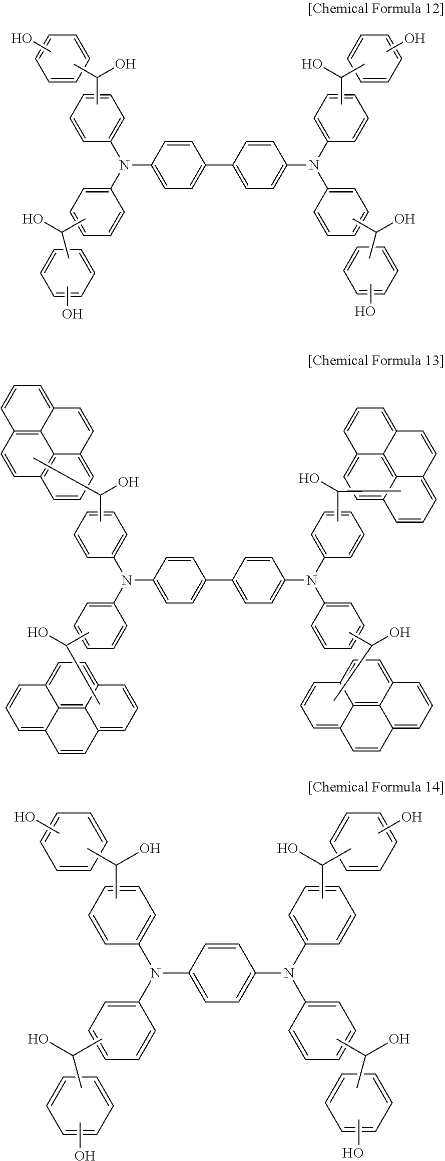

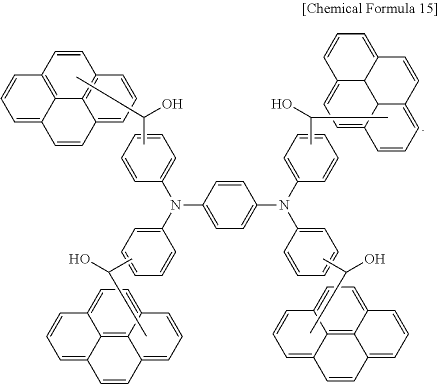

The compound may have a molecular weight of about 500 g/mol to about 10,000 g/mol.

The compound may be included in an amount of about 0.1 wt % to about 30 wt %, based on a total weight of the hardmask composition.

The solvent may include propylene glycol, propylene glycol diacetate, methoxy propanediol, diethylene glycol, diethylene glycol butylether, tri(ethylene glycol)monomethylether, propylene glycol monomethylether, propylene glycol monomethylether acetate, cyclohexanone, ethyl lactate, gamma-butyrolactone, N,N-dimethyl formamide, N,N-dimethyl acetamide, methylpyrrolidone, methylpyrrolidinone, acetylacetone, or ethyl 3-ethoxypropionate.

The embodiments may be realized by providing a hardmask layer including a cured product of the hardmask composition according to an embodiment.

The embodiments may be realized by providing a method of forming patterns, the method including providing a material layer on a substrate; applying the hardmask composition according to an embodiment to the material layer; heat-treating the hardmask composition to form a hardmask layer; forming a photoresist layer on the hardmask layer; exposing and developing the photoresist layer to form a photoresist pattern; selectively removing the hardmask layer using the photoresist pattern to expose a portion of the material layer, and etching an exposed part of the material layer.

Forming the hardmask layer may include heat-treating at about 100° C. to about 1,000° C.

### BRIEF DESCRIPTION OF THE DRAWING

Features will become apparent to those of skill in the art by describing in detail exemplary embodiments with reference to the attached drawing in which:

the FIGURE shows a reference diagram illustrating a level difference of a hardmask layer to explain a method of evaluating planarization characteristics and a calculation formula for evaluating planarization characteristics.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, ‘substituted’ may refer to replacement of a hydrogen atom of a compound by a substituent selected from a halogen atom (F, Br, Cl, or I), a hydroxy group, an alkoxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a vinyl group, a C1 to C20 alkyl group, a C2 to C20 alkenyl group, a C2 to C20 alkynyl group, a C6 to C30 aryl group, a C7 to C30 arylalkyl group, a C9 to C30 allylaryl group, a C1 to C30 alkoxy group, a C1 to C20 heteroalkyl group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, or a combination thereof. As used herein, hydrogen substitution (—H) may include deuterium substitution (-D) or tritium substitution (-T). For example, any hydrogen in any compound described herein may be protium, deuterium, or tritium (e.g., based on natural or artificial substitution). As used herein, the term “or” is not necessarily an exclusive term, e.g., “A or B” would include A, B, or A and B. As used herein, unless described otherwise, * is a linking point.

In addition, adjacent two substituents of the substituted halogen atom (F, Br, Cl, or I), the hydroxy group, the nitro group, the cyano group, the amino group, the azido group, the amidino group, the hydrazino group, the hydrazono group, the carbonyl group, the carbamyl group, the thiol group, the ester group, the carboxyl group or the salt thereof, the sulfonic acid group or the salt thereof, the phosphoric acid or the salt thereof, the C1 to C30 alkyl group, the C2 to C30 alkenyl group, the C2 to C30 alkynyl group, the C6 to C30 aryl group, the C7 to C30 arylalkyl group, the C1 to C30 alkoxy group, the C1 to C20 heteroalkyl group, the C3 to C20 heteroarylalkyl group, the C3 to C30 cycloalkyl group, the C3 to C15 cycloalkenyl group, the C6 to C15 cycloalkynyl group, the C2 to C30 heterocyclic group may be fused to form a ring.

As used herein, when a definition is not otherwise provided, “aromatic hydrocarbon group” refers to a group having one or more hydrocarbon aromatic moieties, including a form in which hydrocarbon aromatic moieties are linked by a single bond, a non-aromatic fused ring form in which hydrocarbon aromatic moieties are fused directly or indirectly, or a combination thereof as well as non-fused aromatic hydrocarbon rings, fused aromatic hydrocarbon rings.

More specifically, the substituted or unsubstituted aromatic hydrocarbon group may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenyl group, a substituted or unsubstituted terphenyl group, a substituted or unsubstituted quaterphenyl group, a substituted or unsubstituted chrysenyl group, a substituted or unsubstituted triphenylenyl group, a substituted or unsubstituted perylenyl group, a substituted or unsubstituted indenyl group, a combination thereof, or a combined fused ring of the foregoing groups, but is not limited thereto.

As used herein, when a definition is not otherwise provided, “hetero” refers to one including 1 to 3 heteroatoms selected from N, O, S, Se, and P.

As used herein, when a definition is not otherwise provided, “heteroaromatic hydrocarbon group” is a concept including a heteroaryl group, and may include at least one heteroatom selected from N, O, S, P, and Si instead of carbon (C) in a cyclic compound such as an aromatic hydrocarbon ring group, a cycloalkyl group, a fused ring thereof, or a combination thereof. When the hetero aromatic hydrocarbon group is a fused ring, the entire ring or each ring of the heterocyclic group may include one or more heteroatoms.

As used herein, when specific definition is not otherwise provided, the term “combination” refers to mixing or copolymerization.

Unless otherwise specified in the present specification, the “molecular weight” is measured by dissolving a powder sample in tetrahydrofuran (THF) and then using 1200 series Gel Permeation Chromatography (GPC) of Agilent Technologies (column is Shodex Company LF-804, standard sample is Shodex company polystyrene).

The hardmask composition according to some embodiments may include a compound represented by Chemical Formula 1, and a solvent.

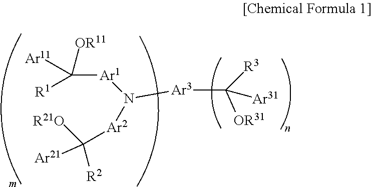

In Chemical Formula 1, Ar¹ to Ar³, Ar¹¹, Ar²¹, and Ar³¹ may each independently be or include, e.g., a substituted or unsubstituted C6 to C30 aromatic hydrocarbon group, a substituted or unsubstituted C3 to C30 hetero aromatic hydrocarbon group, a group in which two or more of the aromatic hydrocarbon rings, heteroaromatic hydrocarbon rings, or a combination thereof are linked by a single bond or a substituted or unsubstituted C1 to C10 alkylene group, or a combination thereof.
R¹ to R³ may each independently be or include, e.g., hydrogen, deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C1 to C30 saturated or unsaturated heteroaliphatic hydrocarbon group, a substituted or unsubstituted C3 to C30 saturated or unsaturated heteroalicyclic hydrocarbon group, a substituted or unsubstituted C6 to C30 aromatic hydrocarbon group, a substituted or unsubstituted C4 to C30 heteroaromatic hydrocarbon group, or a combination thereof.
R¹¹, R²¹, and R³¹ may each independently be or include, e.g., hydrogen, deuterium, a substituted or unsubstituted C1 to C30 saturated or unsaturated aliphatic hydrocarbon group, a substituted or unsubstituted C3 to C30 saturated or unsaturated alicyclic hydrocarbon group, or a substituted or unsubstituted C6 to C30 aromatic hydrocarbon group,
n may be an integer of 0 to 2.
m may be an integer of 1 to 4.

The compound according to some embodiments may include an aromatic hydrocarbon group or a heteroaromatic hydrocarbon group as shown in Chemical Formula 1, and may include a tertiary carbon or quaternary carbon linked thereto, thereby increasing the carbon content in the composition. In addition, a portion of the linking group linking the aromatic hydrocarbon group or heteroaromatic hydrocarbon group may include a nitrogen (N) atom, so that the crosslinking characteristics during curing of the composition may be improved, and the hardmask layer formed therefrom may have excellent etch resistance.

In addition, Chemical Formula 1 may include a tertiary carbon or a quaternary carbon and a substituent represented by —OR¹¹, —OR²¹, or —OR³¹, so that the solubility of the compound in a solvent may be improved, and a hardmask composition containing such a compound may be effectively applied to a spin-on coating process. In addition, since the compound may be crosslinked into a high molecular weight polymer form within a short period of time during heat treatment, the hardmask layer formed therefrom may have excellent heat resistance and excellent etch resistance.

Ar¹ to Ar³ in Chemical Formula 1 may each independently be a substituted or unsubstituted group of a moiety of Group 1.

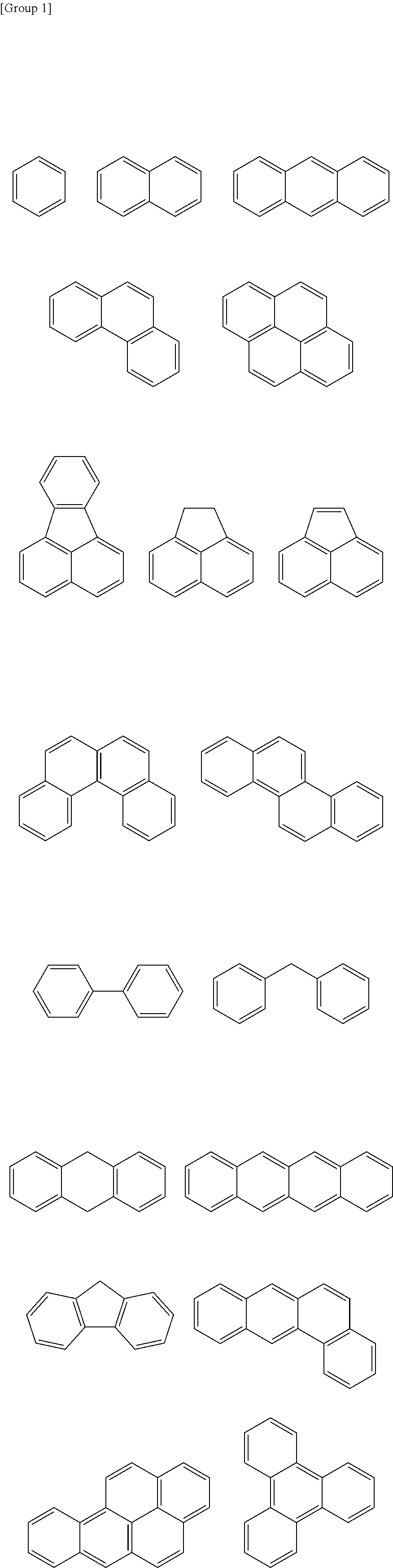

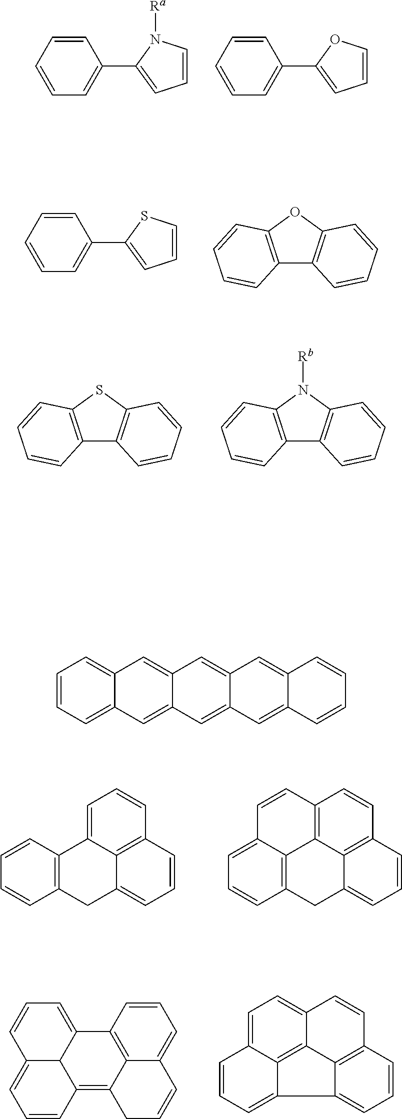

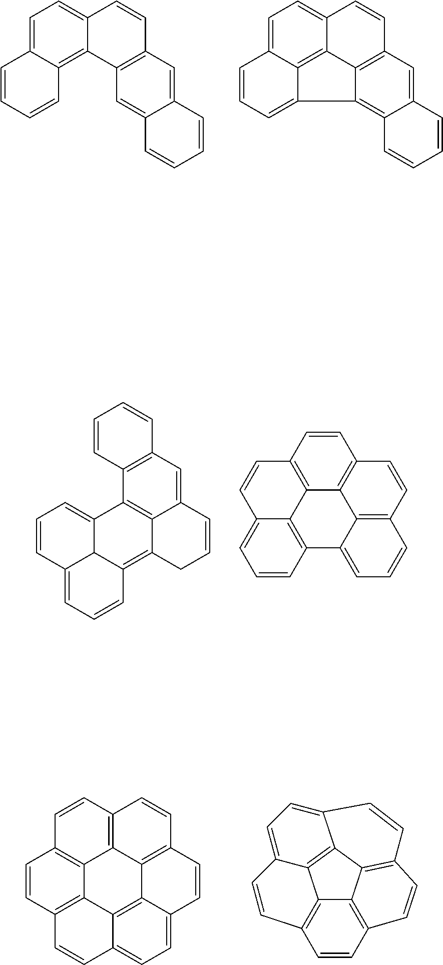

In Group 1, R^{a} and R^{b} may each independently be, e.g., hydrogen, deuterium, a substituted or unsubstituted C1 to C10 alkyl group, or a substituted or unsubstituted C6 to C20 aryl group.

In an implementation, Ar¹ to Ar³ in Chemical Formula 1 may each independently be, e.g., a substituted or unsubstituted group of a moiety of Group 1-1.

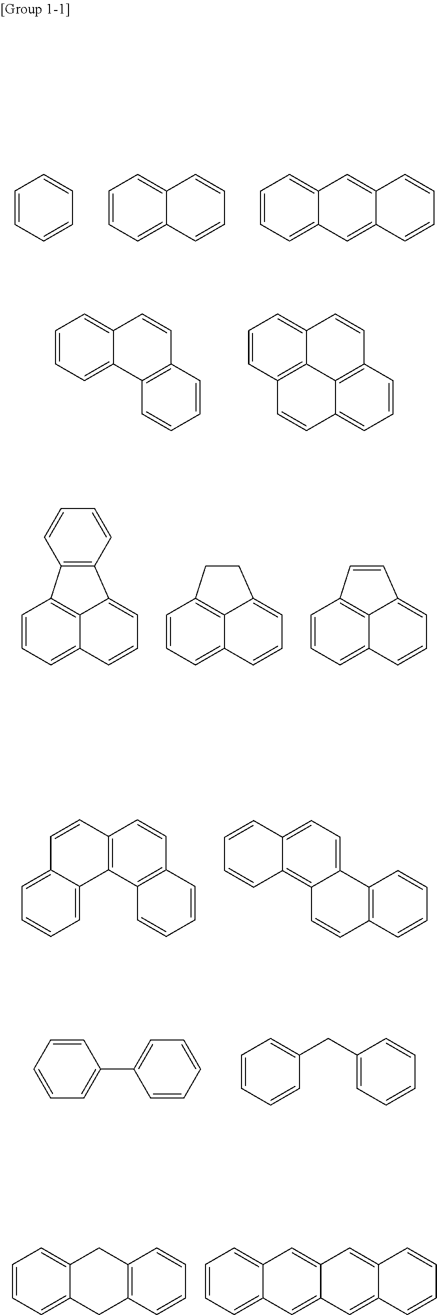

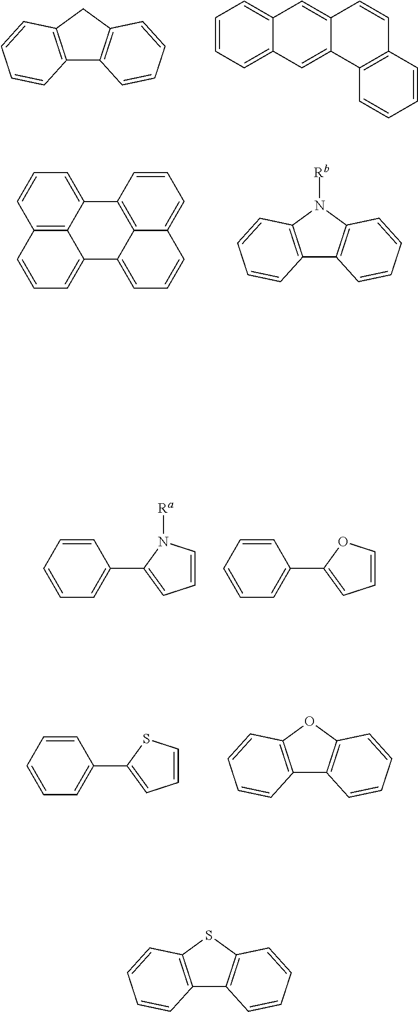

In Group 1-1, R^{a} and R^{b} may each independently be, e.g., hydrogen, deuterium, a substituted or unsubstituted C1 to C10 alkyl group, or a substituted or unsubstituted C6 to C20 aryl group.

In an implementation, Ar¹ to Ar³ in Chemical Formula 1 may each independently be, e.g., a substituted or unsubstituted group of a moiety of Group 1-2.

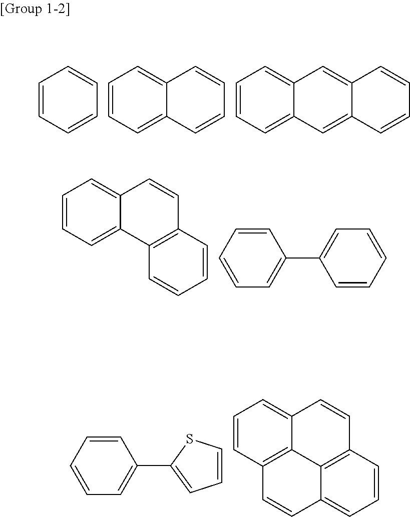

In an implementation, in Chemical Formula 1, Ar¹¹, Ar²¹, and Ar³¹ may each independently be, e.g., a substituted or unsubstituted C6 to C30 aromatic hydrocarbon group. In an implementation, Ar¹¹, Ar²¹, and Ar³¹ in Chemical Formula 1 may each independently be a substituted or unsubstituted group of a moiety of Group 2. In an implementation, Ar¹¹, Ar²¹, and Ar³¹ in Chemical Formula 1 may each independently be a substituted or unsubstituted group of a moiety of Group 2-1.

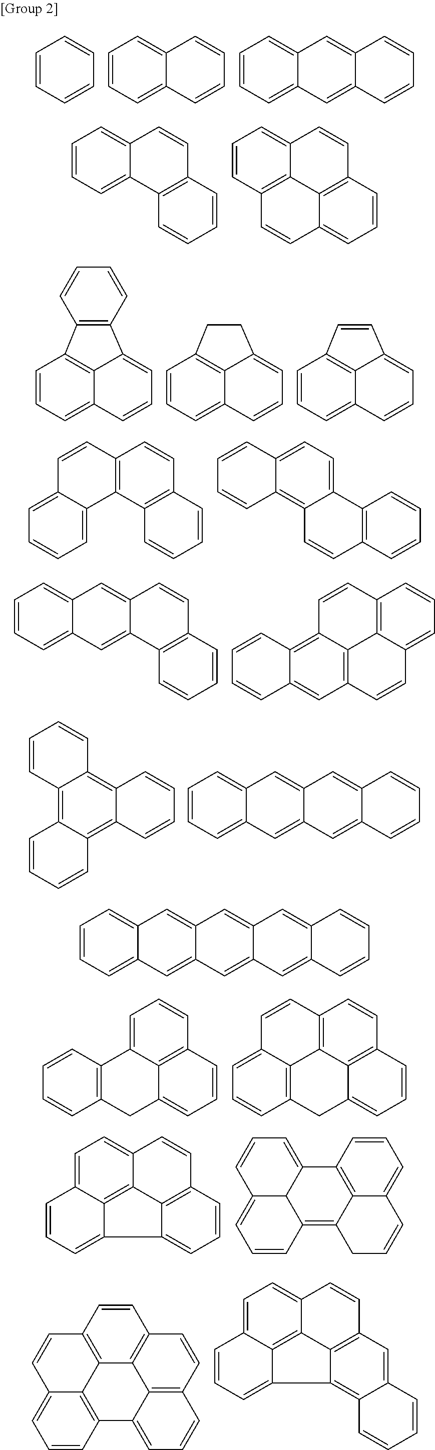

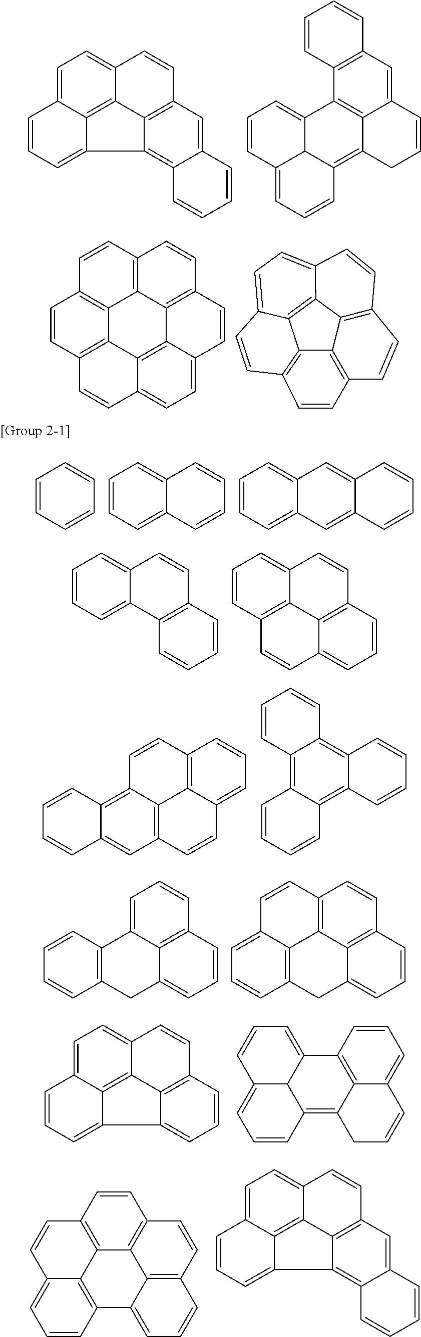

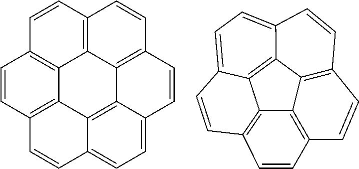

In an implementation, Ar¹ to Ar³ in Chemical Formula 1 may each independently be a substituted or unsubstituted group of a moiety of Group 1-2, and Ar¹¹, Ar²¹, and Ar³¹ may each independently be a substituted or unsubstituted group of a moiety of Group 2-1.

In an implementation, R¹ to R³ in Chemical Formula 1 may each independently be, e.g., hydrogen, deuterium, a substituted or unsubstituted C1 to C30 saturated or unsaturated aliphatic hydrocarbon group, a substituted or unsubstituted C6 to C30 aromatic hydrocarbon group, or a combination thereof,

In an implementation, R¹¹, R²¹, and R³¹ in Chemical Formula 1 may each independently be, e.g., hydrogen, deuterium, or a substituted or unsubstituted C1 to C30 saturated or unsaturated aliphatic hydrocarbon group.

In an implementation, R¹ to R³, R¹¹, R²¹, and R³¹ in Chemical Formula 1 may each independently be, e.g., hydrogen, deuterium, a substituted or unsubstituted C1 to C20 alkyl group, a substituted or unsubstituted C6 to C20 aromatic hydrocarbon group, or a combination thereof. In an implementation, R¹ to R³, R¹¹, R²¹, and R³¹ in Chemical Formula 1 may each independently be, e.g., hydrogen, deuterium, a methyl group, an ethyl group, a propyl group, a butyl group, a phenyl group, a naphthyl group, an anthryl group, a phenanthryl group, or a combination thereof. In an implementation, R¹ to R³, R¹¹, R²¹, and R³¹ in Chemical Formula 1 may each independently be, e.g., hydrogen, deuterium, a methyl group, a phenyl group, or a combination thereof.

In an implementation, in Chemical Formula 1, n may be an integer of 0 to 2, e.g., 0, or 1.

In an implementation, in Chemical Formula 1, m may be an integer of 1 to 4. In an implementation, in Chemical Formula 1, m may be an integer of 1 to 3, e.g., 1 or 2.

In an implementation, in Chemical Formula 1, each of Ar¹ and Ar², Ar¹¹ and Ar²¹, R¹ and R², or R¹¹ and R²¹ may be the same, only some of them may be the same, or each may be different from each other. That Ar¹ and Ar², Ar¹¹ and Ar²¹, R¹ and R², or R¹¹ and R²¹ may each be the same may mean that Ar¹ and Ar² may be the same, Ar¹ and Ar²¹ may be the same, R¹ and R² may be the same, and R¹¹ and R²¹ may be the same.

In an implementation, in Chemical Formula 1, Ar³ may be the same as or different from Ar¹ or Ar².

In an implementation, if n in Chemical Formula 1 may be 1 or more, Ar¹¹ may be the same as Ar¹¹ or Ar²¹, R³ may be the same as R¹ or R², and R³¹ may be the same as R¹¹ or R²¹.

In an implementation, if m in Chemical Formula 1 may be 2 or more, each of Ar¹, Ar², Ar¹¹, Ar²¹, R¹, R², R¹¹, and R²¹ may be the same or different from each other.

In an implementation, the compound represented by Chemical Formula 1 may be represented by one of Chemical Formula 2 to Chemical Formula 15.

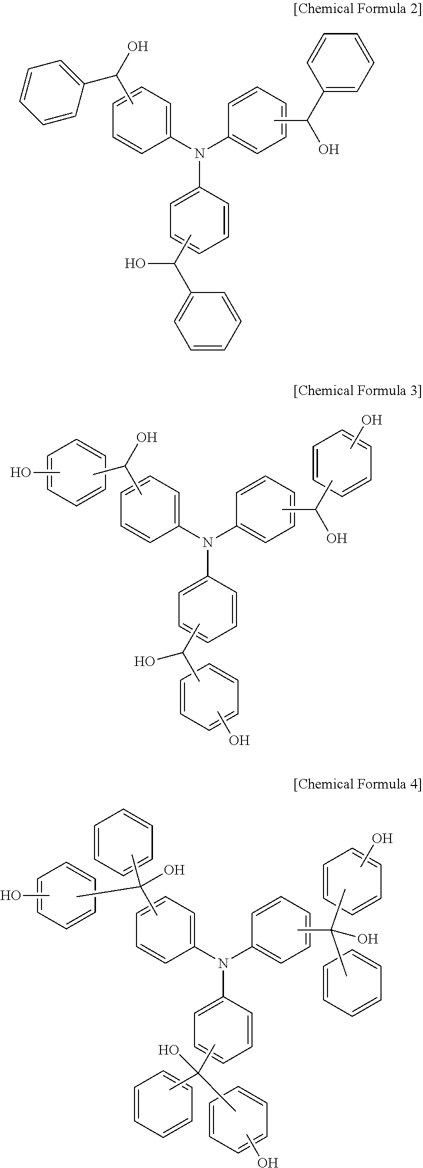

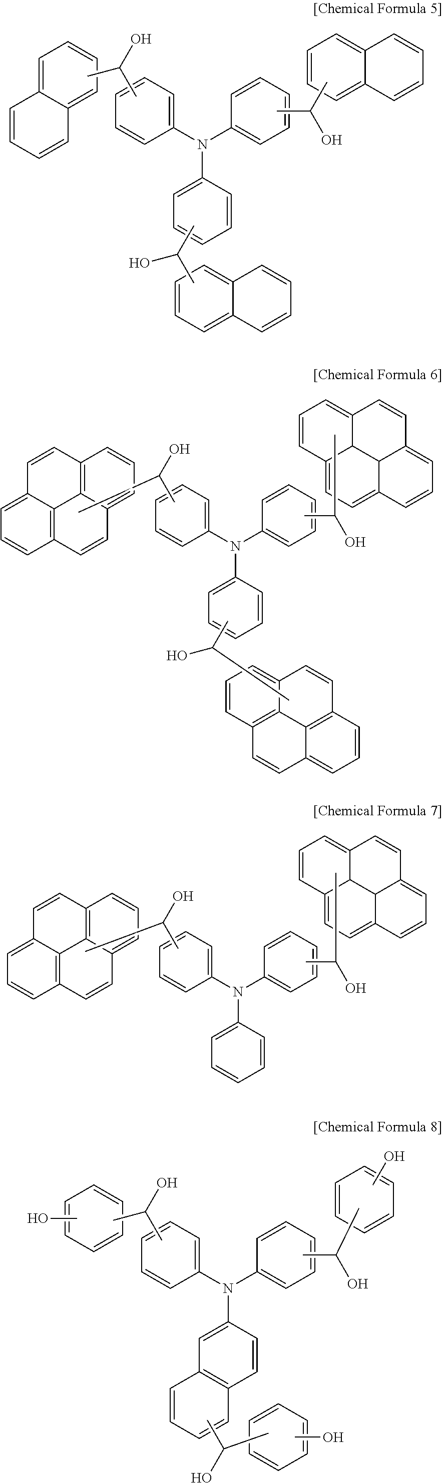

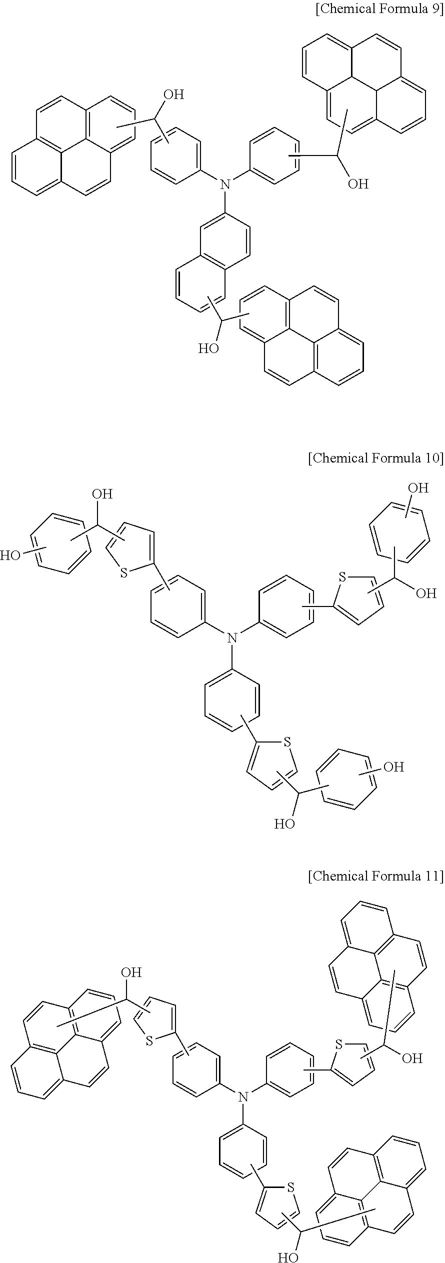

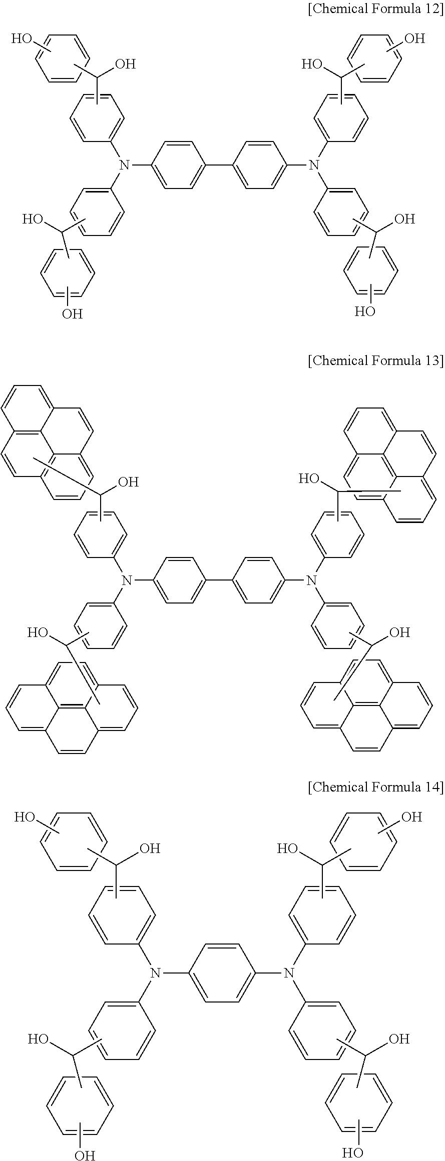

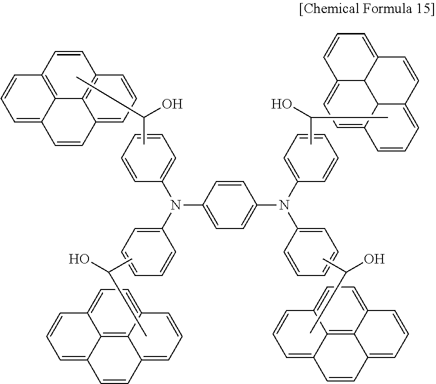

The compound may have a molecular weight of, e.g., about 500 g/mol to about 10,000 g/mol. In an implementation, the compound may have a molecular weight, e.g., of about 500 g/mol to about 9,500 g/mol, about 500 g/mol to about 9,000 g/mol, about 600 g/mol to about 8,500 g/mol, about 600 g/mol to about 8,000 g/mol, about 700 g/mol to about 7,500 g/mol, or about 700 g/mol to about 7,000 g/mol. By having a molecular weight within the above ranges, the carbon content and solubility in the solvent of the hardmask composition including the above compound may be adjusted and optimized.

The compound may be included in an amount of, e.g., about 0.1 wt % to about 30 wt % based on a total weight of the hardmask composition. In an implementation, the compound may be included in an amount of, e.g., about 0.2 wt % to about 30 wt %, about 0.5 wt % to about 30 wt %, about 1 wt % to about 30 wt %, about 1 wt % to about 25 wt %, or about 1 wt % to about 20 wt %. By including the compound within the above ranges, a thickness, a surface roughness, and a planarization degree of the hardmask may be easily adjusted.

The hardmask composition may include a solvent. In an implementation, the solvent may include, e.g., propylene glycol, propylene glycol diacetate, methoxy propanediol, diethylene glycol, diethylene glycol butyl ether, tri(ethylene glycol) monomethyl ether, propylene glycol monomethyl ether, propylene glycol monomethyl ether acetate, cyclohexanone, ethyl lactate, gamma-butyrolactone, N,N-dimethylformamide, N,N-dimethylacetamide, methylpyrrolidone, methylpyrrolidinone, acetylacetone, ethyl 3-ethoxypropionate, or the like. In an implementation, the solvent may be a suitable solvent that has sufficient solubility or dispersibility for the compound.

In an implementation, the hardmask composition may further include an additive, e.g., a surfactant, a crosslinking agent, a thermal acid generator, or a plasticizer.

The surfactant may include, e.g., a fluoroalkyl-based compound, alkylbenzene sulfonate, alkylpyridinium salt, polyethylene glycol, a quaternary ammonium salt, and the like.

The crosslinking agent may be, e.g., a melamine, a substituted urea, or a polymer crosslinking agent. In an implementation, it may be a crosslinking agent having at least two crosslinking substituents, e.g., methoxymethylated glycoruryl, butoxymethylated glycoruryl, methoxymethylated melamine, butoxymethylated melamine, methoxymethylated benzoguanamine, butoxy methylated benzoguanamine, methoxymethylated urea, butoxymethylated urea, methoxymethylated thiourea, or butoxymethylated thiourea.

In an implementation, as the crosslinking agent, a crosslinking agent having high heat resistance may be used. The crosslinking agent having high heat resistance may include a compound containing a crosslinking substituent having an aromatic ring (e.g., a benzene ring or a naphthalene ring) in the molecule.

In an implementation, the thermal acid generator may be, e.g., an acid compound, such as p-toluenesulfonic acid, trifluoromethanesulfonic acid, pyridinium p-toluenesulfonic acid, salicylic acid, sulfosalicylic acid, citric acid, benzoic acid, hydroxybenzoic acid, naphthalenecarboxylic acid or 2,4,4,6-tetrabromocyclohexadienone, benzointosylate, 2-nitrobenzyltosylate, or other organic sulfonic acid alkyl esters.

According to some embodiments, a hardmask layer including a cured product of the aforementioned hardmask composition may be provided.

Hereinafter, a method of forming patterns using the aforementioned hardmask composition may be described.

A method of forming patterns according to some embodiments may include providing a material layer on a substrate, applying a hardmask composition including the aforementioned compound and solvent to the material layer, heat-treating the hardmask composition to form a hardmask layer, forming a photoresist layer on the hardmask layer, exposing and developing the photoresist layer to form a photoresist pattern, selectively removing the hardmask layer using the photoresist pattern to expose a part of the material layer, and etching the exposed part of the material layer.

The substrate may be, e.g., a silicon wafer, a glass substrate, or a polymer substrate. The material layer may be a material to be finally patterned, e.g., a metal layer such as an aluminum layer or a copper layer, a semiconductor layer such as a silicon layer, or an insulation layer such as a silicon oxide layer or a silicon nitride layer. The material layer may be formed through a method such as a chemical vapor deposition (CVD) process.

The hardmask composition may be the same as described above, and may be applied by spin-on coating in a form of a solution. In an implementation, an application thickness of the hardmask composition may be, e.g., about 50 Å to about 200,000 Å.

The heat-treating of the hardmask composition may be performed, e.g., at about 100° C. to about 1,000° C. for about 10 seconds to about 1 hour. In an implementation, the heat-treating of the hardmask composition may include a plurality of heat-treating processes, e.g., a first heat-treating process, and a second heat-treating process.

In an implementation, the heat-treating of the hardmask composition may include, e.g., one heat-treating process performed at about 100° C. to about 1,000° C. for about 10 seconds to about 1 hour, and, e.g., the heat-treating may be performed under an atmosphere of air or nitrogen, or an atmosphere having oxygen concentration of 1 wt % or less.

In an implementation, the heat-treating of the hardmask composition may include, e.g., a first heat-treating process performed at about 100° C. to about 1,000° C., about 100° C. to about 800° C., about 100° C. to about 500° C., or about 150° C. to about 400° C. for about 30 seconds to about 1 hour, e.g. about 30 seconds to about 30 minutes, about 30 seconds to about 10 minutes, or about 30 seconds to about 5 minutes.

In an implementation, the heat-treating of the hardmask composition may include, e.g., a second heat-treating process performed at about 100° C. to 1,000° C., about 300° C. to about 1,000° C., about 500° C. to about 1,000° C., or about 500° C. to about 600° C. for about 30 seconds to about 1 hour, e.g., about 30 seconds to about 30 minutes, about 30 seconds to about 10 minutes, or about 30 seconds to about 5 minutes, consecutively. In an implementation, the first and second heat-treating process may be performed under an air or nitrogen atmosphere, or may be performed under an atmosphere with an oxygen concentration of 1 wt % or less.

By performing at least one of the steps of heat-treating the hardmask composition at a high temperature, e.g., of 200° C. or higher, high etch resistance capable of withstanding etching gas and chemical liquid exposed in subsequent processes including the etching process may be exhibited.

In an implementation, the forming of the hardmask layer may include a UV/Vis curing process/or a near IR curing process.

In an implementation, the forming of the hardmask layer may include a first heat-treating process, a second heat-treating process, a UV/Vis curing process, or a near IR curing process, or may include two or more processes consecutively.

In an implementation, the method may further include forming a silicon-containing thin layer on the hardmask layer. The silicon-containing thin layer may include, e.g., SiCN, SiOC, SiON, SiOCN, SiC, SiO, SiN, or the like.

In an implementation, the method may further include forming a bottom antireflective coating (BARC) on the silicon-containing thin layer or on the hardmask layer before forming the photoresist layer.

In an implementation, exposure of the photoresist layer may be performed using, e.g., ArF, KrF, or EUV. After exposure, heat-treating may be performed at about 100° C. to about 700° C.

In an implementation, the etching process of the exposed part of the material layer may be performed through a dry etching process using an etching gas and the etching gas may be, e.g., N₂/O₂, CHF₃, CF₄, Cl₂, BCl₃, or a mixed gas thereof.

The etched material layer may be formed in a plurality of patterns, and the plurality of patterns may be a metal pattern, a semiconductor pattern, an insulation pattern, or the like, e.g., diverse patterns of a semiconductor integrated circuit device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Synthesis of Compounds

### Synthesis Example 1

9.94 g of triphenylamine, 17.37 g of benzoyl chloride, and 132.93 g of 1,2-dichloroethane were added to the flask to prepare a mixed solution. 19.77 g of aluminum chloride was slowly added to the mixed solution at ambient temperature and stirred for 10 hours. When a reaction was completed, the reaction mixture was washed with water and concentrated under reduced pressure, and n-hexane was added thereto. The solid generated at this time was filtered out and dried under reduced pressure to obtain Intermediate Product A-1 represented by Chemical Formula A-1.

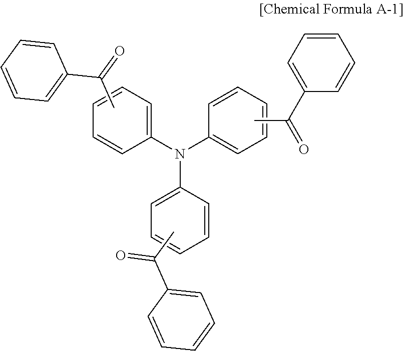

In a flask, 25.15 g of Intermediate Product A-1 and 150.84 g of N-methylpyrrolidinone were added to prepare a mixed solution. To the mixed solution, 10.24 g of sodium borohydride was slowly added at ambient temperature and then, heated to 60° C. and stirred, until Intermediate Product A-1 was all reacted. After the stirring, the resultant was neutralized to pH 7 by using a 1% hydrogen chloride solution and washed and concentrated under a reduced pressure, and n-hexane was added thereto. Herein, a solid produced therein was filtered and dried under the reduced pressure to obtain Compound 1 represented by Chemical Formula A.

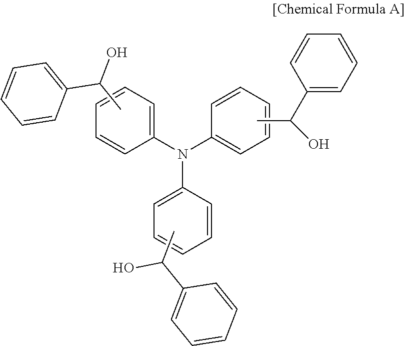

### Synthesis Example 2

In a flask, 9.21 g of triphenylamine, 19.54 g of 4-methoxybenzoyl chloride, and 132.93 g of 1,2-dichloroethane were added to prepare a mixed solution. To the mixed solution, 18.32 g of aluminum chloride was slowly added at ambient temperature and then, stirred for 10 hours. When a reaction was completed, the resultant was washed and concentrated under a reduced pressure, and n-hexane was added thereto. Herein, a solid produced therein was filtered and dried under a reduced pressure to obtain Intermediate Product B-1 represented by Chemical Formula B-1.

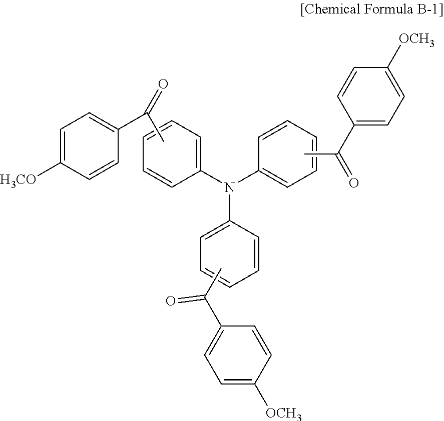

In a flask, 13.00 g of Intermediate Product B-1, 16.25 g of 1-dodecanethiol, 6.76 g of potassium hydroxide, and 144.00 g of N-methylpyrrolidinone were added to prepare a mixed solution. The mixed solution was heated to 90° C. and stirred until Intermediate Product B-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Intermediate Product B-2 represented by Chemical Formula B-2.

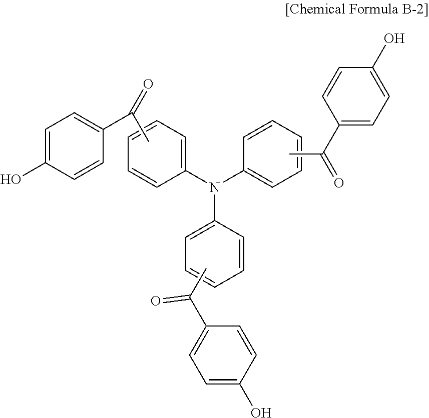

In a flask, 25.15 g of Intermediate Product B-2 and 150.84 g of N-methylpyrrolidinone were added to prepare a mixed solution. To the mixed solution, 10.24 g of sodium borohydride was slowly added at ambient temperature and then, heated to 60° C. and stirred, until Intermediate Product B-2 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 2 represented by Chemical Formula B.

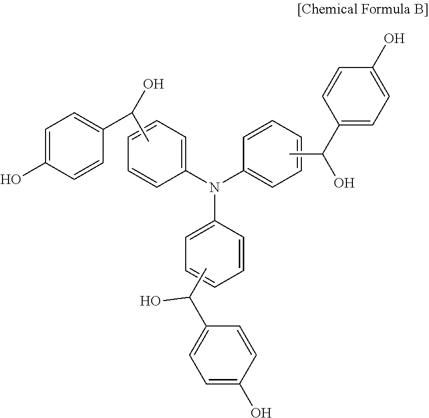

### Synthesis Example 3

In a flask, 15.49 g of Intermediate Product B-2 of Synthesis Example 2 and 93.74 g of tetrahydrofuran were added to prepare a mixed solution. To the mixed solution, 90.77 g of a 27% phenylmagnesium chloride tetrahydrofuran solution was slowly added at ambient temperature and then, heated to 60° C. and stirred until Intermediate Product B-2 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 3 represented by Chemical Formula C.

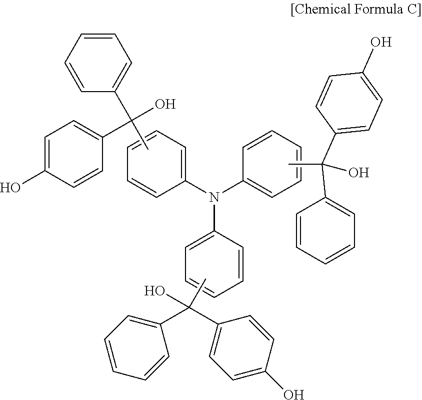

### Synthesis Example 4

In a flask, 8.78 g of triphenylamine, 20.82 g of 2-naphthoyl chloride, and 132.93 g of 1,2-dichloroethane were added to prepare a mixed solution. The mixed solution was stirred for 10 hours, while 17.47 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product D-1 represented by Chemical Formula D-1.

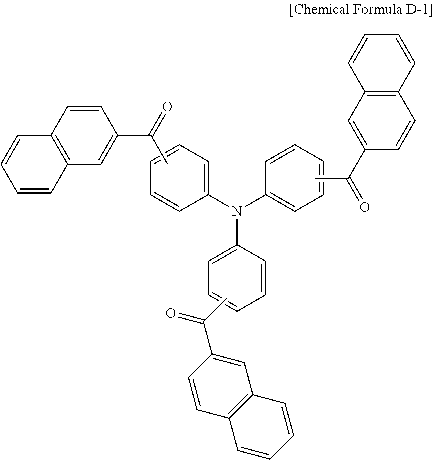

In a flask, 26.79 g of Intermediate Product D-1 and 150.84 g of N-methylpyrrolidinone were added to prepare a mixed solution. To the mixed solution, 8.59 g of sodium borohydride was slowly added at ambient temperature and then, heated at 60° C. and stirred until Intermediate Product D-1 was all used. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 4 represented by Chemical Formula D.

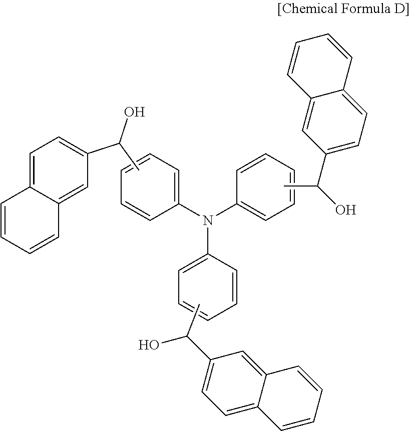

### Synthesis Example 5

In a flask, 7.46 g of triphenylamine, 24.76 g of 1-pyrenecarbonyl chloride, and 132.93 g of 1,2-dichloroethane were added to prepare a mixed solution. The mixed solution was stirred for 10 hours, while 14.85 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product E-1 re resented by Chemical Formula E-1.

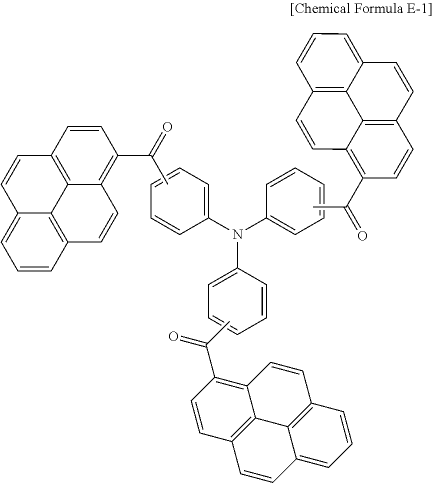

In a flask, 28.44 g of Intermediate Product E-1 and 150.84 g of N-methylpyrrolidinone were added to prepare a mixed solution. To the mixed solution, 6.94 g of sodium borohydride was slowly added at ambient temperature and then, heated to 60° C. and stirred until Intermediate Product E-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 5 represented by Chemical Formula E.

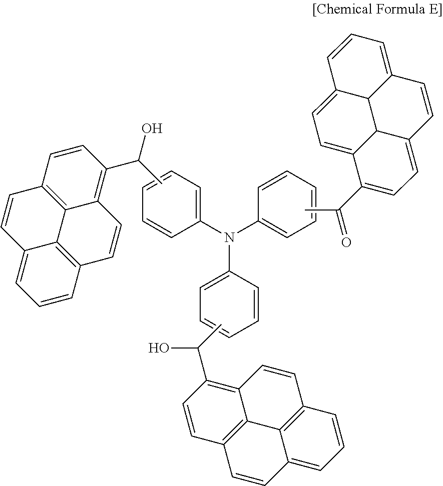

### Synthesis Example 6

In a flask, 10.88 g of triphenylamine, 26.03 g of 1-pyrenecarbonyl chloride, and 147.70 g of 1,2-dichloroethane were added to prepare a mixed solution. The mixed solution was stirred for 10 hours, while 15.38 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product F-1 represented by Chemical Formula F-1.

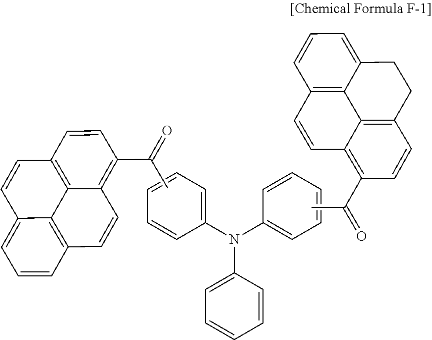

In a flask, 15.11 g of Intermediate Product F-1 and 80.00 g of N-methylpyrrolidinone were added to prepare a mixed solution. To the mixed solution, 4.89 g of aluminum borohydride was slowly added at ambient temperature and then, heated to 60° C. and stirred until Intermediate Product F-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 6 represented by Chemical Formula F.

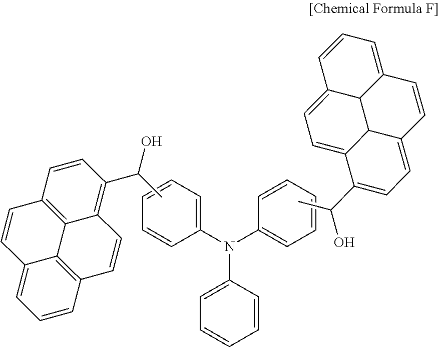

### Synthesis Example 7

In a flask, 7.46 g of triphenylamine, 10.66 g of N,N-diphenyl-2-naphthylamine, 18.79 g of 4-methoxybenzoyl chloride, and 132.93 g of 1,2-dichloroethane were added to prepare a mixed solution. The mixed solution was stirred for 10 hours, while 17.62 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product G-1 represented by Chemical Formula G-1.

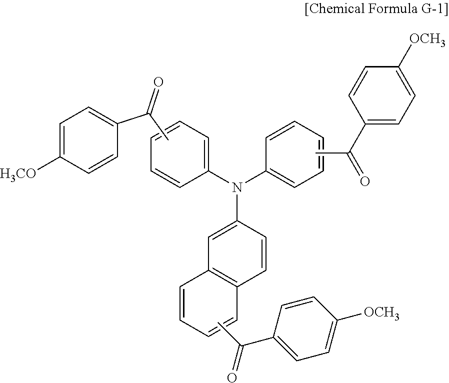

In a flask, 7.46 g of triphenylamine, 9.27 g of Intermediate Product G-1, 18.88 g of 1-dodecanethiol, 7.85 g of potassium hydroxide, and 144.00 g of N-methylpyrrolidinone were added to prepare a mixed solution. The mixed solution was heated to 90° C. and then, stirred, until Intermediate Product G-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Intermediate Product G-2 represented by Chemical Formula G-2.

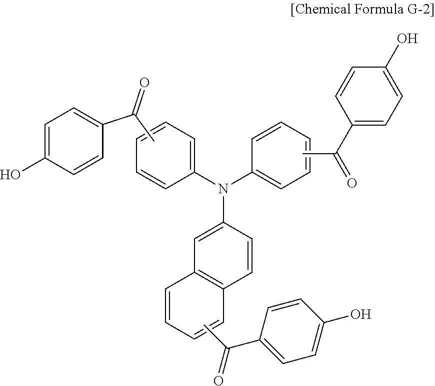

In a flask, 7.46 g of triphenylamine, 25.20 g of Intermediate Product G-2 and 150.84 g of N-methylpyrrolidinone were added to prepare a mixed solution. To the mixed solution, 10.18 g of sodium borohydride was slowly added at ambient temperature and then, heated to 60° C. and stirred, until Intermediate Product G-2 was all reacted. After the stirring, the resultant was neutralized to pH7 by using a 1% hydrogen chloride solution, washed, and concentrated under a reduced pressure, and n-hexane was added thereto. Herein, a solid produced therein was filtered and then, dried under a reduced pressure to obtain Compound 7 represented by Chemical Formula G.

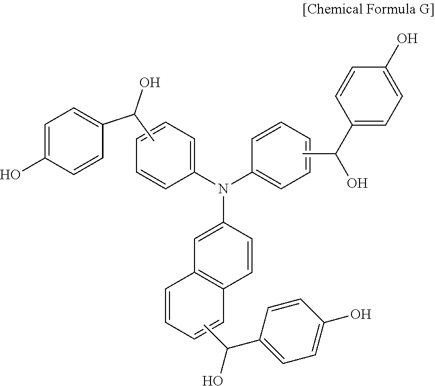

### Synthesis Example 8

In a flask, 7.46 g of triphenylamine, 8.71 g of N,N-diphenyl-2-naphthylamine, 23.98 g of 1-pyrenecarbonyl chloride, and 132.93 g of 1,2-dichloroethane were added to prepare a mixed solution. The mixed solution was stirred for 10 hours, while 14.38 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product H-1 represented by Chemical Formula H-1.

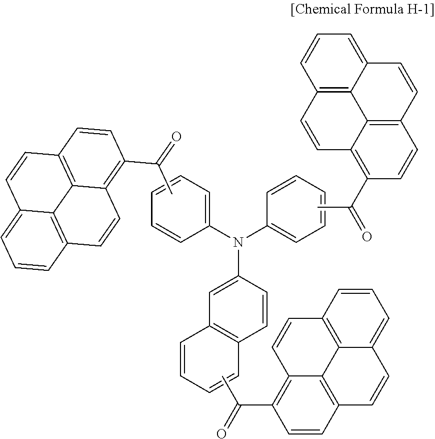

In a flask, 7.46 g of triphenylamine, 28.73 g of Intermediate Product H-1 and 150.84 g of N-methylpyrrolidinone were added to prepare a mixed solution. To the mixed solution, 6.65 g of sodium borohydride was slowly added at ambient temperature and then, heated to 60° C., until Intermediate Product H-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 8.

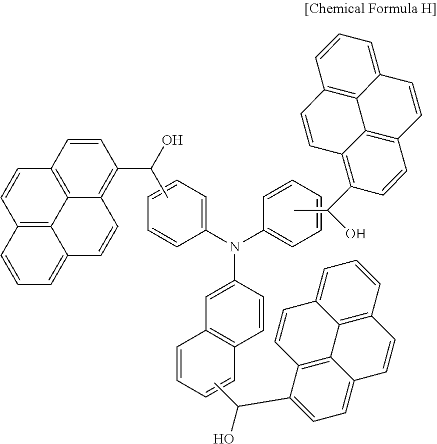

### Synthesis Example 9

In a flask, 15.43 g of tris[4-(2-thienyl)phenyl]amine, 16.33 g of 4-methoxybenzoyl chloride, and 132.93 g of 1,2-dichloroethane were added to prepare a mixed solution. The mixed solution was stirred for 10 hours, while 15.31 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product I-1 represented by Chemical Formula I-1.

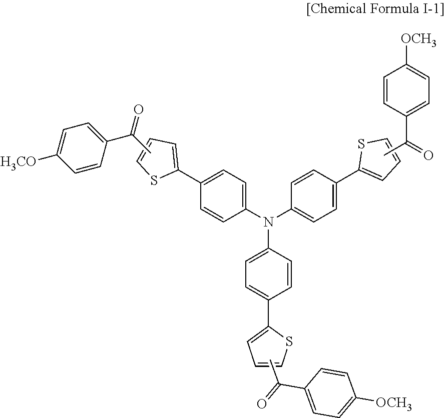

In a flask, 7.46 g of triphenylamine, 15.78 g of Intermediate Product I-1, 14.28 g of 1-dodecanethiol, 5.94 g of potassium hydroxide, and 144.00 g of N-methylpyrrolidinone were added to prepare a mixed solution. The mixed solution was heated to 90° C. and stirred, until Intermediate Product I-1 was all used. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Intermediate Product I-2 represented by Chemical Formula I-2.

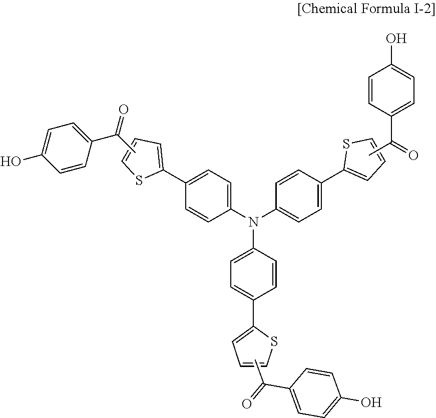

In a flask, 26.99 g of Intermediate Product I-2 and 150.84 g of N-methylpyrrolidinone were added to prepare a mixed solution. To the mixed solution, 8.39 g of sodium borohydride was slowly added at ambient temperature and then, heated to 60° C. and stirred until Intermediate Product I-2 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 9 represented by Chemical Formula I.

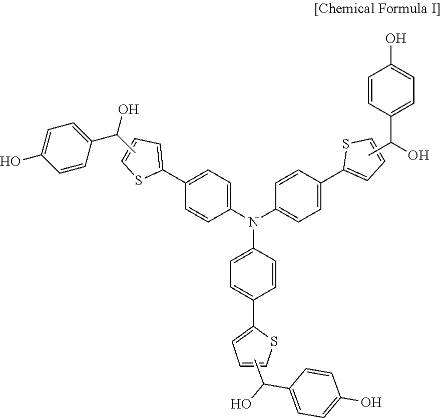

### Synthesis Example 10

In a flask, 7.46 g of triphenylamine, 12.91 g of tris[4-(2-thienyl)phenyl]amine, 21.35 g of 1-pyrenecarbonyl chloride, and 132.93 g of 1,2-dichloroethane were added to prepare a mixed solution. The mixed solution was stirred for 10 hours, while 12.81 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product J-1 represented by Chemical Formula J-1.

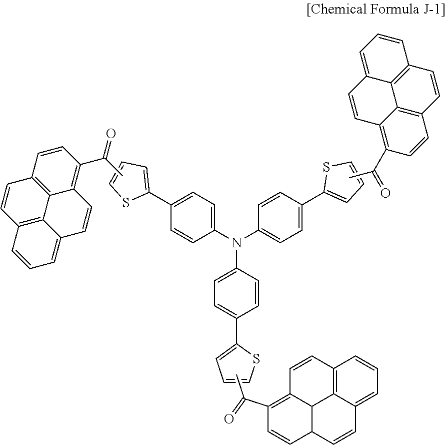

In a flask, 31.35 g of Intermediate Product J-1, 24.76 g of 1-pyrenecarbonyl chloride and 150.84 g of N-methylpyrrolidinone were added to prepare a mixed solution. To the mixed solution, 4.03 g of sodium borohydride was slowly added at ambient temperature and then, heated to 60° C. and stirred, until Intermediate Product J-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 10 represented by Chemical Formula J.

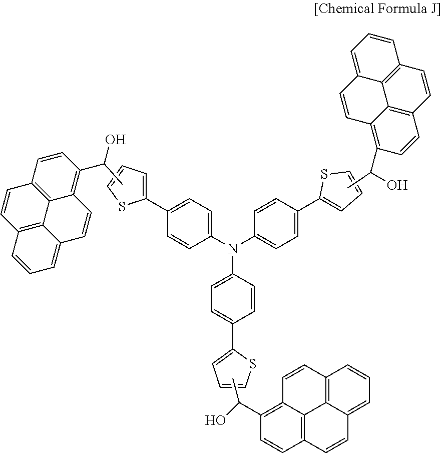

### Synthesis Example 11

In a flask, 12.40 g of N,N,N,N-tetraphenylbenzidine, 7.75 g of 4-methoxybenzoyl chloride, and 132.93 g of 1,2-dichloroethane were added to prepare a mixed solution. The mixed solution was stirred for 10 hours, while 16.92 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product K-1 represented by Chemical Formula K-1.

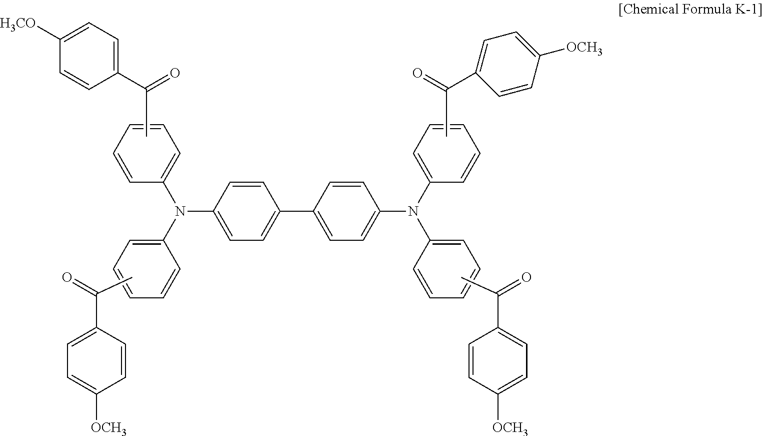

In a flask, 7.46 g of triphenylamine, 15.55 g of Intermediate Product K-1, 15.35 g of 1-dodecanethiol, 5.11 g of potassium hydroxide, and 144.00 g of N-methylpyrrolidinone were added to prepare a mixed solution. The mixed solution was heated to 90° C. and stirred until Intermediate Product K-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Intermediate Product K-2 represented by Chemical Formula K-2.

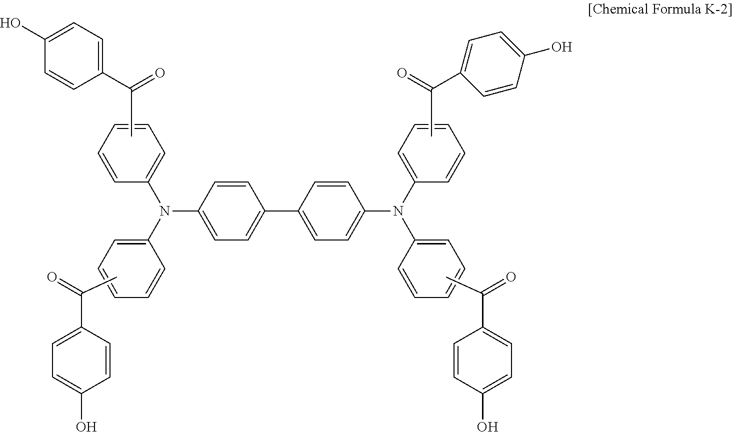

In a flask, 26.18 g of Intermediate Product K-2 and 150.84 g of N-methylpyrrolidinone were added to prepare a mixed solution. To the mixed solution, 9.20 g of sodium borohydride was added and then, heated to 60° C. and stirred until Intermediate Product K-2 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 11 represented by Chemical Formula K.

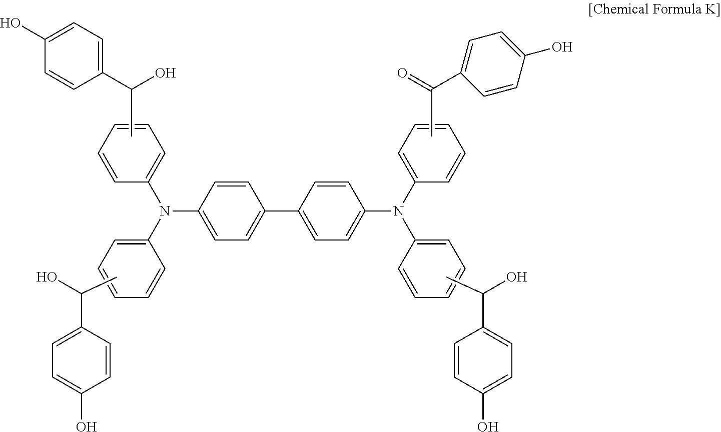

### Synthesis Example 12

In a flask, 10.23 g of N,N,N,N-tetraphenylbenzidine, 22.89 g of 1-pyrenecarbonyl chloride, and 132.93 g of 1,2-dichloroethane were added to prepare a mixed solution. The mixed solution was stirred for 10 hours, while 13.95 g of aluminum chloride was slowly added thereto at ambient temperature. When a reaction was completed, the resultant was washed and concentrated under a reduced pressure, and n-hexane was added thereto. Herein, a solid produced therein was filtered and then, dried under a reduced pressure to obtain Intermediate Product L-1 represented by Chemical Formula L-1.

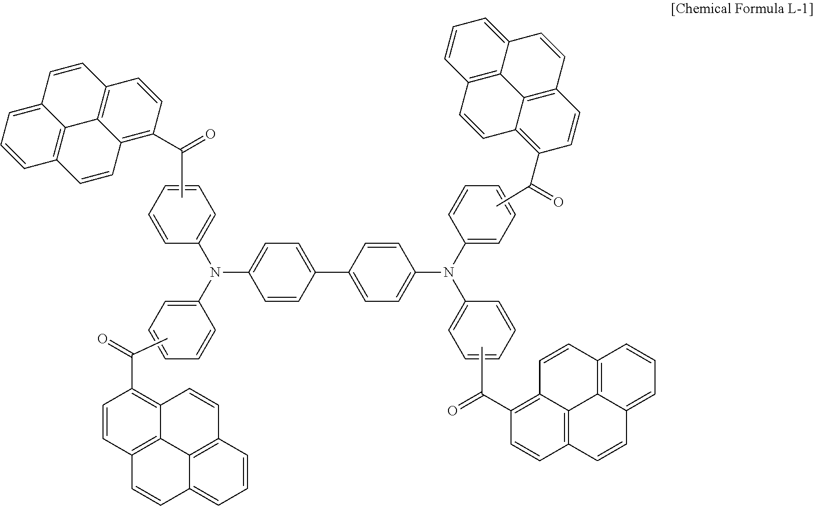

In a flask, 31.17 g of Intermediate Product L-1 and 150.84 g of N-methylpyrrolidinone were added to prepare a mixed solution. To the mixed solution, 4.21 g of sodium borohydride was slowly added at ambient temperature and then, heated to 60° C. and stirred until Intermediate Product L-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 12 represented by Chemical Formula L.

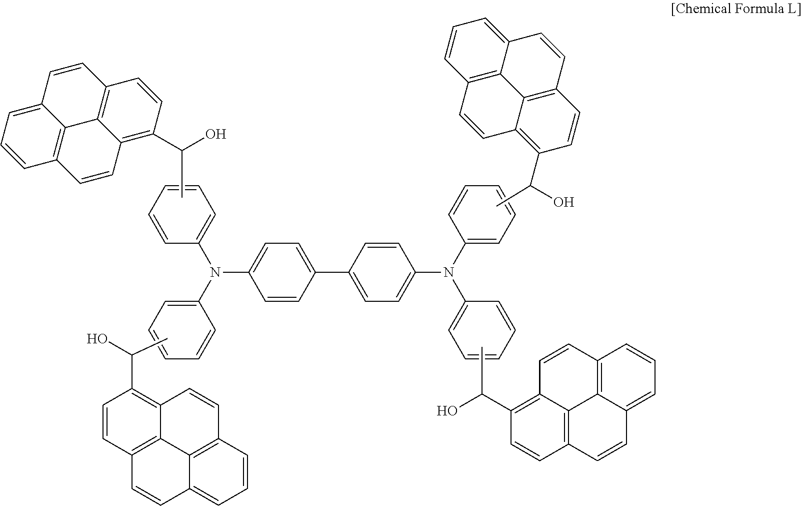

### Synthesis Example 13

In a flask, 10.92 g of N,N,N,N-tetraphenyl-1,4-phenylenediamine, 18.51 g of 4-methoxybenzoyl chloride, and 132.93 g of 1,2-dichloroethane were added to prepare a mixed solution. The mixed solution was stirred for 10 hours, while 17.64 g of aluminum chloride was slowly added thereto at ambient temperature. When a reaction was completed, the resultant was washed and concentrated under a reduced pressure, and n-hexane was added thereto. Herein, a solid produced therein was filtered and then, dried under a reduced pressure to obtain Intermediate Product M-1 represented by Chemical Formula M-1.

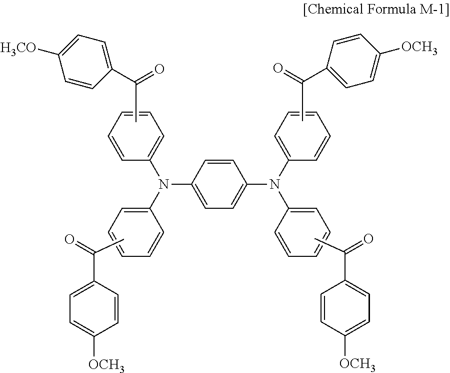

In a flask, 14.87 g of Intermediate Product M-1, 15.86 g of 1-dodecanethiol, 5.27 g of potassium hydroxide, and 144.00 g of N-methylpyrrolidinone were added to prepare a mixed solution. The mixed solution was heated to 90° C. and stirred until Intermediate Product M-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Intermediate Product M-2 represented by Chemical Formula M-2.

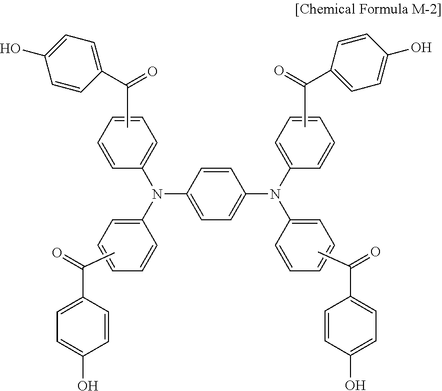

In a flask, 29.20 g of Intermediate Product M-2 and 150.84 g of N-methylpyrrolidinone were added to prepare a mixed solution. To the mixed solution, 6.18 g of sodium borohydride was slowly added at ambient temperature and then, heated to 60° C. and stirred until Intermediate Product M-2 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 13 represented by Chemical Formula M.

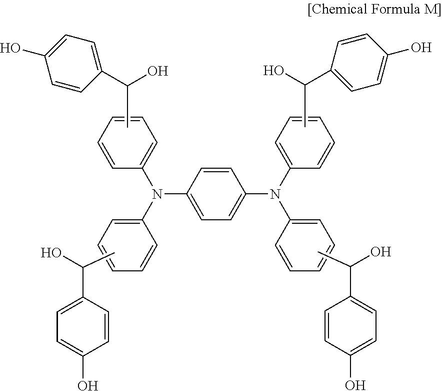

### Synthesis Example 14

In a flask, 8.94 g of N,N,N,N-tetraphenyl-1,4-phenylenediamine, 23.69 g of 1-pyrenecarbonyl chloride, and 132.93 g of 1,2-dichloroethane were added to prepare a mixed solution. The mixed solution was stirred for 10 hours, while 14.44 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product N-1 represented by Chemical Formula N-1.

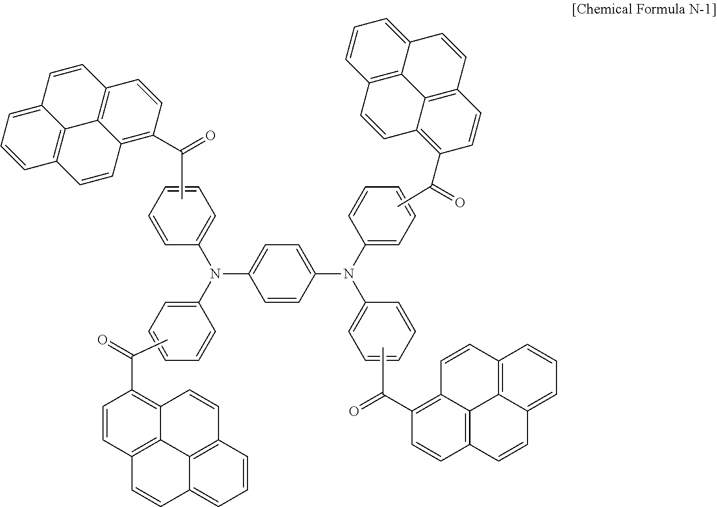

In a flask, 30.96 g of Intermediate Product N-1 and 150.84 g of N-methylpyrrolidinone were added to prepare a mixed solution. To the mixed solution, 14.85 g of aluminum chloride was slowly added at ambient temperature and then, heated to 60° C. and stirred, until the Intermediate Product N-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Compound 14 represented by Chemical Formula N.

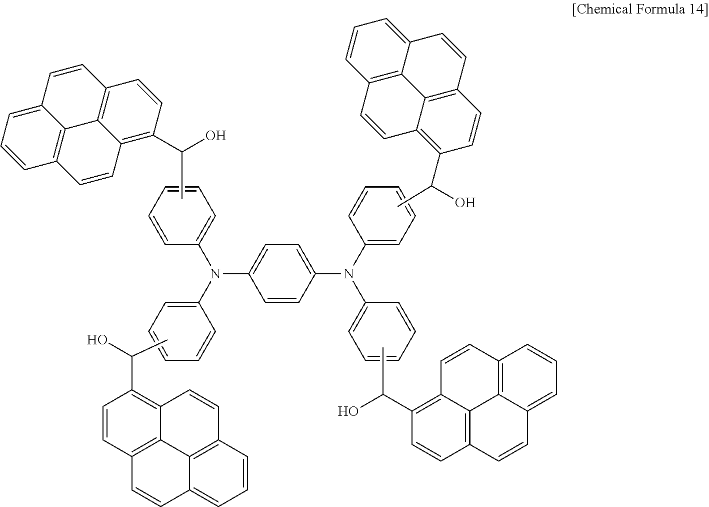

### Comparative Synthesis Example

In a flask, 21.68 g of terephthaloyl chloride, 49.37 g of 1-methoxypyrene, and 399.89 g of 1,2-dichloroethane were added to prepare a mixed solution. The mixed solution was stirred for 10 hours, while 29.05 g of aluminum chloride was slowly added at ambient temperature. When a reaction was completed, the resultant was washed and concentrated under a reduced pressure, and n-hexane was added thereto. Herein, a solid produced therein was filtered and then, dried under a reduced pressure to obtain Intermediate Product P-1 represented by Chemical Formula P-1.

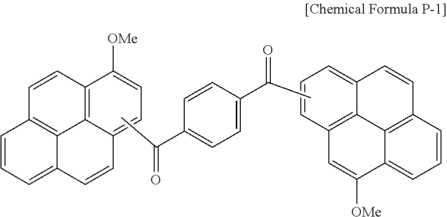

In a flask, 25.02 g of Intermediate Product P-1, 18.88 g of 1-dodecanethiol, 9.44 g of potassium hydroxide, and 240.00 g of N-methylpyrrolidinone were added to prepare a mixed solution. The mixed solution was heated to 90° C. and stirred, until Intermediate Product P-1 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Intermediate Product P-2 represented by Chemical Formula P-2.

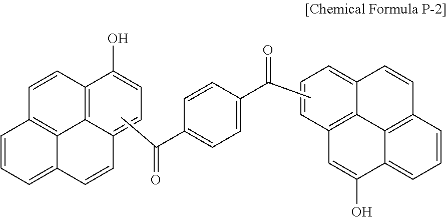

In a flask, 16.22 g of Intermediate Product P-2 and 125.63 g of N-methylpyrrolidinone were added to prepare a mixed solution. To the mixed solution, 8.67 g of sodium borohydride was slowly added at ambient temperature and then, heated to 60° C. and stirred until Intermediate Product P-2 was all reacted. After the stirring, the resultant was neutralized to pH 7 by using a 1% hydrogen chloride solution, washed, and concentrated under a reduced pressure, and n-hexane was added thereto. Herein, a solid produced therein was filtered and then, dried under a reduced pressure to obtain Comparative Compound P represented by Chemical Formula P.

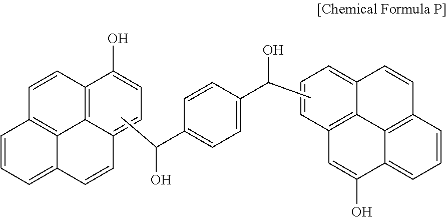

### Preparation of Hardmask Compositions

### Examples and Comparative Examples

Each of the compounds and the comparative compounds of Synthesis Examples 1 to 14 and the comparative synthesis example were dissolved in cyclohexanone and then, filtered with a membrane filter having a diameter of 0.1 μm to prepare a hardmask composition having each compound content of 10.0 wt %.

### Evaluation 1: Heat Resistance

Each of the hardmask compositions of Examples 1 to 14 and the Comparative Example were spin-coated on a silicon wafer and then, heat-treated on a hot plate at 160° C. for 1 minute to form a hardmask layer. A thickness of the hardmask layer was measured by using a film thickness meter made by K-MAC Co., Ltd. Subsequently, the hardmask layer was reheat-treated at 400° C. for 2 minutes to remeasure a thickness of the hardmask layer. If the hardmask layer had a thickness variation ratio of less than 5% before and after the reheat-treatment at 400° C. for 2 minutes, “A” (very good) was given, greater than or equal to 5% and less than 10%, “B” (good) was given, and greater than or equal to 10%, “C” (inferior) was given. The results are shown in Table 1.

TABLE 1

| | Heat resistance |
|---|---|
| Example 1 | A |
| Example 2 | A |
| Example 3 | A |
| Example 4 | A |
| Example 5 | A |
| Example 6 | A |
| Example 7 | A |
| Example 8 | A |
| Example 9 | A |
| Example 10 | A |
| Example 11 | A |
| Example 12 | A |
| Example 13 | A |
| Example 14 | A |
| Comparative Example | C |

Referring to Table 1, the hardmask layers formed of the compositions of Examples 1 to 14 exhibited a thickness variation ratio of less than 5% before and after the reheat treatment, and the hardmask layer formed of the composition according to the Comparative Example exhibited a thickness variation ratio of greater than or equal to 10% before and after the reheat treatment. In other words, the hardmask layers formed of the compositions of Examples 1 to 14 exhibited excellent heat resistance, compared to the hardmask layer formed of the composition according to the Comparative Example.

### Evaluation 2: Gap-Fill Characteristics

Each of the hardmask compositions Examples 1 to 14 and the Comparative Example were spin-coated on a patterned silicon wafer and then, heat-treated at 400° C. for 60 seconds to form a hardmask layer. A pattern cross-section image of the hardmask layer was examined with a field emission scanning electron microscope (FE-SEM) to check whether or not voids occurred. The results are shown in Table 2.

TABLE 2

| | Whether voids occurred |
|---|---|
| Example 1 | Voids did not occur |
| Example 2 | Voids did not occur |
| Example 3 | Voids did not occur |
| Example 4 | Voids did not occur |
| Example 5 | Voids did not occur |
| Example 6 | Voids did not occur |
| Example 7 | Voids did not occur |
| Example 8 | Voids did not occur |
| Example 9 | Voids did not occur |
| Example 10 | Voids did not occur |
| Example 11 | Voids did not occur |
| Example 12 | Voids did not occur |
| Example 13 | Voids did not occur |
| Example 14 | Voids did not occur |
| Comparative Example | Voids did not occur |

Referring to Table 2, no voids were generated in the hardmask layer formed from the hardmask compositions according to Examples 1 to 14, and gap-fill characteristics of the hardmask layer formed from the hardmask compositions according to the examples were improved.

### Evaluation 3: Planarization Characteristics

Each of the hardmask compositions of Examples 1 to 14 and the Comparative Example were respectively spin-coated on a patterned silicon wafer and heat-treated at 400° C. for 60 seconds, and a pattern cross-section image thereof was examined through a field emission scanning electron microscope (FE-SEM). A thickness of the hardmask layer shown in the pattern cross-section image was measured, which was used to digitalize planarization degrees according to calculation equation of the FIGURE. In the FIGURE, h₁ refers to an average value of the thickness of the hardmask layer measured at three arbitrary points where a pattern was not formed, and h₂ refers to an average value of the thickness of the hardmask layer measured at three arbitrary points where a pattern was formed. If the numerical results of planarization degrees were less than 5, “A” (very good) was given, if they were between 5 and 10, “B” (good) was given, and if they were over 10, “C” (inferior) was given. The results are shown in Table 3.

TABLE 3

| | Planarization characteristics |
|---|---|
| Example 1 | A |
| Example 2 | A |
| Example 3 | A |
| Example 4 | A |
| Example 5 | A |
| Example 6 | A |
| Example 7 | A |
| Example 8 | A |
| Example 9 | A |
| Example 10 | A |
| Example 11 | A |
| Example 12 | A |
| Example 13 | A |
| Example 14 | A |
| Comparative Example | C |

Referring to Table 3, the planarization degrees of the hardmask layer formed from the hardmask compositions according to Examples 1 to 14 were very good, while the planarization degree of the hardmask layer formed from the composition according to the Comparative Example was deteriorated.

### Evaluation 4: Etch Resistance

Each of the hardmask compositions of Examples 1 to 14 and the Comparative Example were spin-coated on a silicon wafer and then, heat-treated on a hot plate at 160° C. for 1 minute to form a hardmask layers. The hardmask layers was dry etched by using CF₄/Ar mixed gas for 30 seconds and then, measured with respect to a hardmask thickness before and after the etching by using a film thickness meter made by K-MAC Co., Ltd. to calculate an etch rate (bulk etch rate, BER) according to Calculation Equation 2. Etch resistance was evaluated as a ratio of the etch rate of the hardmask layer formed from the compositions according to Examples 1 to 14 to the etch rate of the hardmask layer formed from the composition according to the Comparative Example. If the ratio was less than 0.95, it was evaluated as “A” (very good), if it was between 0.95 and less than 1.00, it was evaluated as “B” (good), and if it was 1.00 or more, it was evaluated as “C” (inferior). The results are shown in Table 4.

Etch rate (Å/s)=(Initial thin film thickness−Thin film thickness after etching)/Etching time (s) [Calculation Equation 2]

TABLE 4

| | Etch resistance |
|---|---|
| Example 1 | A |
| Example 2 | A |
| Example 3 | A |
| Example 4 | A |
| Example 5 | A |
| Example 6 | A |
| Example 7 | A |
| Example 8 | A |
| Example 9 | A |
| Example 10 | A |
| Example 11 | A |
| Example 12 | A |
| Example 13 | A |
| Example 14 | A |

Referring to Table 4, the etch rates of the hardmask layers formed from the compositions according to Examples 1 to 14 were less than 0.95 compared to the etch rate of the hardmask layer formed from the composition according to the Comparative Example, indicating that the etch resistance of the hardmask layers formed from the compositions according to Examples 1 to 14 were superior to that of the hardmask layer formed from the composition according to the Comparative Example.

By way of summation and review, there is a constant trend in the semiconductor industry to reduce a size of chips. To respond to this, the line width of the resist patterned in lithography technology may have a size of several tens of nanometers. Therefore, a height that can withstand the line width of the resist pattern may be limited, and there may be cases where the resists may not have sufficient resistance to withstand the etching step. In order to compensate for this, an auxiliary layer, which is called a hardmask layer, may be used between a material layer to be etched and a photoresist layer. This hardmask layer may serve as an interlayer that transfers a fine pattern of the photoresist through selective etching. Therefore, the hardmask layer may be required to have sufficient etch resistance as to withstand the etching process during the pattern transfer.

Some hardmask layers may be formed in a chemical or physical deposition method and may have low economic efficiency due to a large-scale equipment and a high process cost. Accordingly, a spin-coating technique for forming a hardmask layer has recently been developed. The spin-coating technique may be an easier process to conduct than the conventional method, and a hardmask layer formed therefrom may exhibit much more excellent gap-fill characteristics and planarization characteristics. However, the hardmask layer formed using the spin-coating technique may have a required etch resistance is somewhat lowered. Accordingly, a hardmask composition may be required to apply to the spin-coating technique and to secure equivalent etch resistance to that of the hardmask layer formed in the chemical or physical deposit method.

In order to improve the etch resistance of a hardmask layer, research on maximizing a carbon content of the hardmask composition is being made. However, as a carbon content of the compound included in the hardmask composition is maximized, solubility of the compound in a solvent may decrease. A carbon content of the compound included in the hardmask composition may be maximized to improve the etch resistance of the hardmask layer formed therefrom, while the compound may be well soluble in the solvent.

The hardmask composition according to some embodiments may include a compound having an aromatic hydrocarbon group or a heteroaromatic hydrocarbon group, thereby increasing the carbon content in the composition. Accordingly, the hardmask layer obtained from the composition may secure excellent etch resistance. In addition, the compound may include specific functional groups, thereby increasing the carbon content in the composition and ensuring excellent solubility in solvents.

One or more embodiments may provide a hardmask composition that can be effectively applied to a hardmask layer.

One or more embodiments may provide a hardmask layer including a cured product of the hardmask composition.

One or more embodiments may provide a method of forming patterns using the hardmask composition.

The hardmask composition according to some embodiments may have excellent solubility in solvents and may be effectively applied to a hardmask layer.

The hardmask composition according to some embodiments may have excellent crosslinking characteristics when cured, and the hardmask layer formed therefrom may secure excellent heat resistance and excellent etch resistance.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A hardmask composition, comprising:
a compound represented by Chemical Formula 1;
and a solvent, 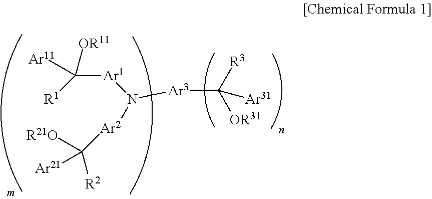
wherein, in Chemical Formula 1,
Ar¹ to Ar³, Ar¹¹, Ar²¹, and Ar³¹ are each independently a substituted or unsubstituted C6 to C30 aromatic hydrocarbon group, a substituted or unsubstituted C3 to C30 hetero aromatic hydrocarbon group, a group in which two or more of the aromatic hydrocarbon groups, heteroaromatic hydrocarbon groups, or a combination thereof are linked by a single bond or a substituted or unsubstituted C1 to C10 alkylene group, or a combination thereof,
R¹ to R³ are each independently hydrogen, deuterium, a substituted or unsubstituted C1 to C30 saturated or unsaturated aliphatic hydrocarbon group, a substituted or unsubstituted C3 to C30 saturated or unsaturated alicyclic hydrocarbon group, a substituted or unsubstituted C1 to C30 saturated or unsaturated heteroaliphatic hydrocarbon group, a substituted or unsubstituted C3 to C30 saturated or unsaturated heteroalicyclic hydrocarbon group, a substituted or unsubstituted C6 to C30 aromatic hydrocarbon group, a substituted or unsubstituted C4 to C30 heteroaromatic hydrocarbon group, or a combination thereof,
R¹¹, R²¹, and R³¹ are each independently hydrogen, deuterium, a substituted or unsubstituted C1 to C30 saturated or unsaturated aliphatic hydrocarbon group, a substituted or unsubstituted C3 to C30 saturated or unsaturated alicyclic hydrocarbon group, or a substituted or unsubstituted C6 to C30 aromatic hydrocarbon group,
n is an integer of 0 to 2, and
m is an integer of 1 to 4.

2. The hardmask composition as claimed in claim 1, wherein:
in Chemical Formula 1, Ar¹ to Ar³ are each independently a substituted or unsubstituted group of a moiety of Group 1: 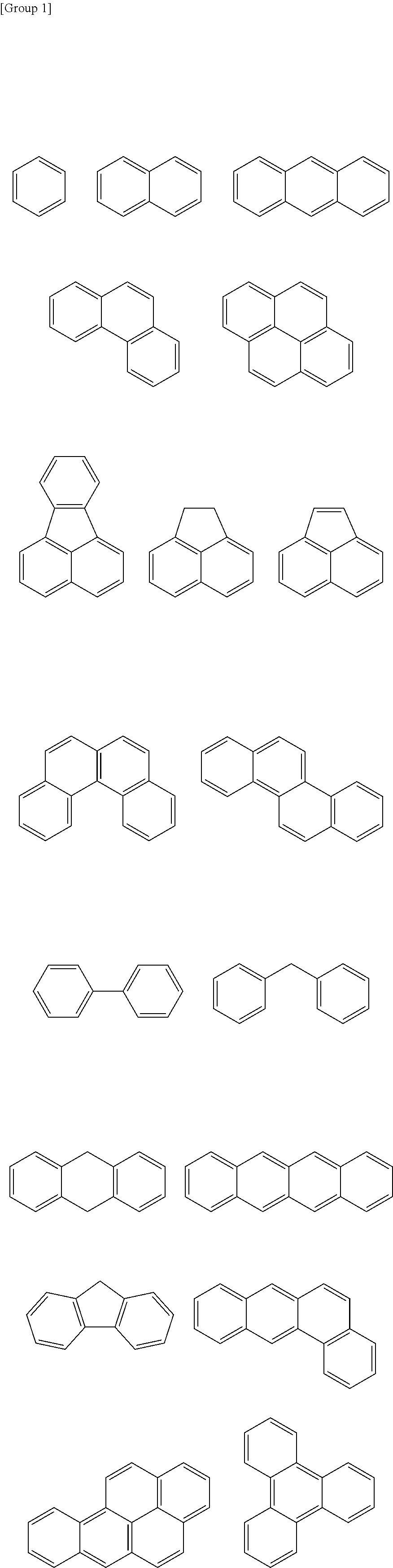 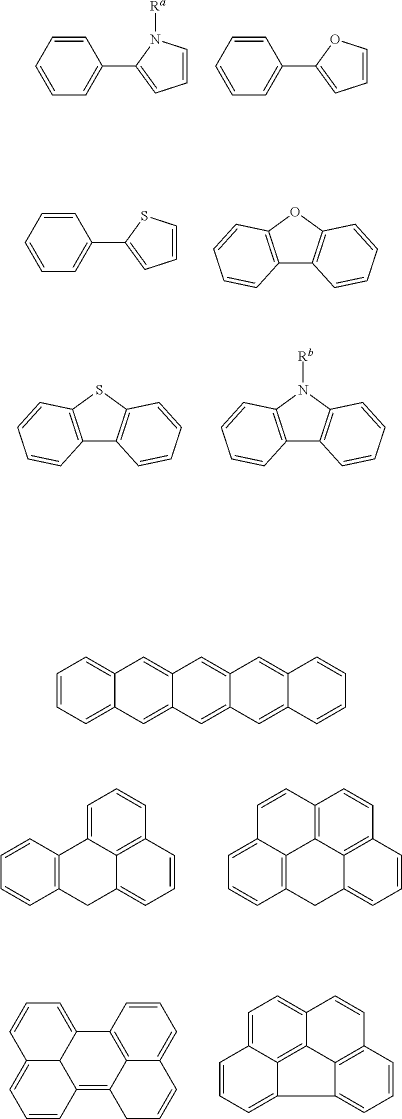 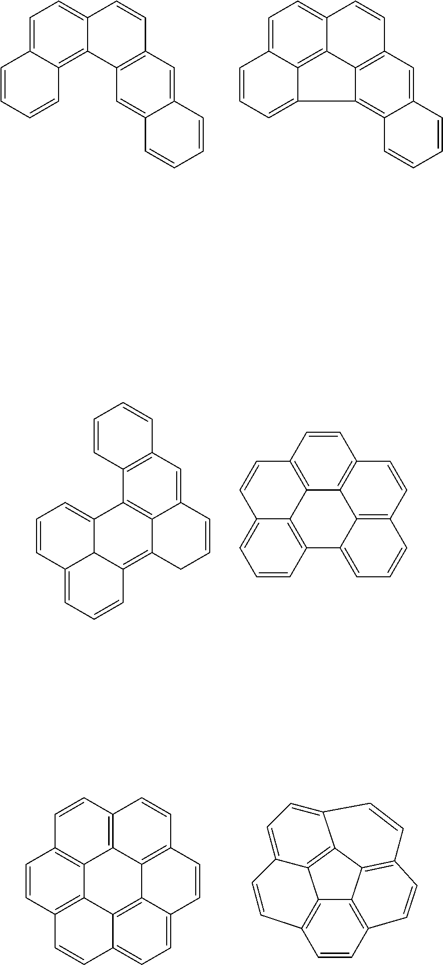
in Group 1, R^{a} and R^{b} are each independently hydrogen, deuterium, a substituted or unsubstituted C1 to C10 alkyl group, or a substituted or unsubstituted C6 to C20 aryl group.

3. The hardmask composition as claimed in claim 1, wherein:
in Chemical Formula 1, Ar¹ to Ar³ are each independently a substituted or unsubstituted group of a moiety of Group 1-1: 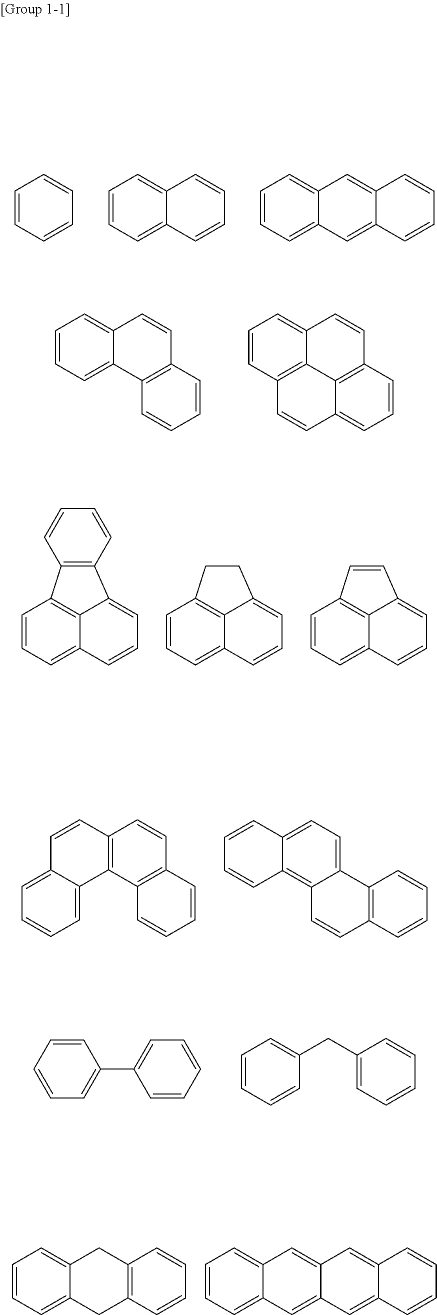 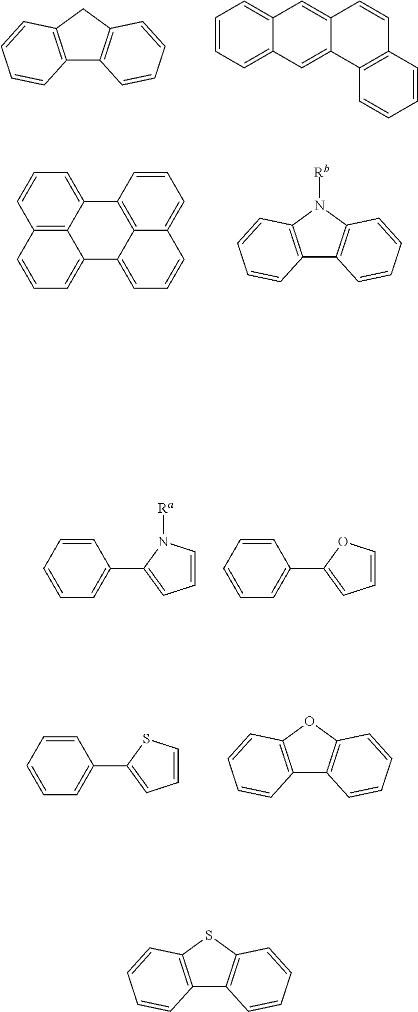
in Group 1-1, R^{a} and R^{b} are each independently hydrogen, deuterium, a substituted or unsubstituted C1 to C10 alkyl group, or a substituted or unsubstituted C6 to C20 aryl group.

4. The hardmask composition as claimed in claim 1, wherein in Chemical Formula 1, Ar¹¹, Ar²¹, and Ar³¹ are each independently a substituted or unsubstituted C6 to C30 aromatic hydrocarbon group.

5. The hardmask composition as claimed in claim 1, wherein in Chemical Formula 1, Ar¹¹, Ar²¹, and Ar³¹ are each independently a substituted or unsubstituted group of a moiety of Group 2:

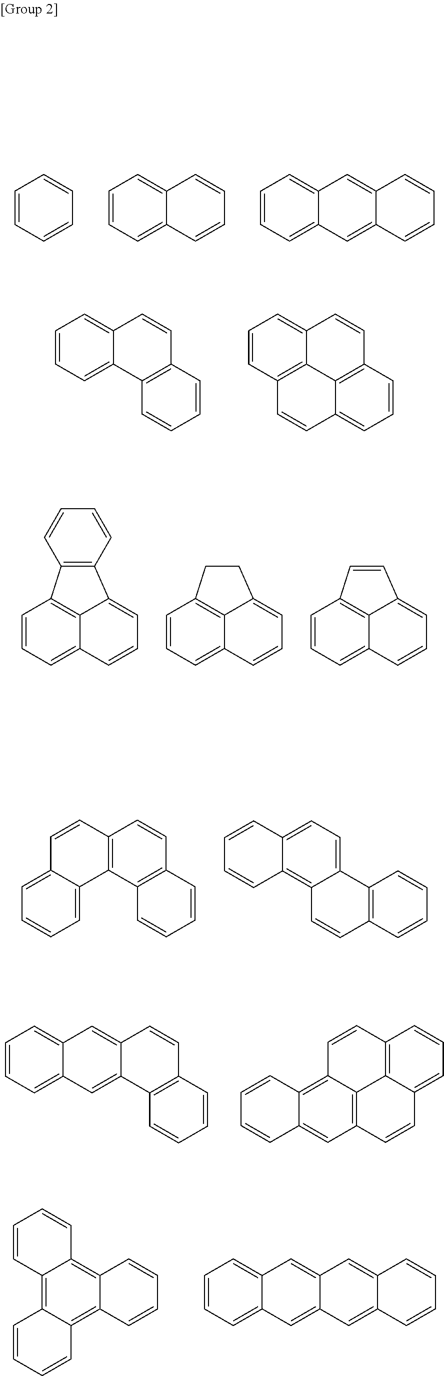

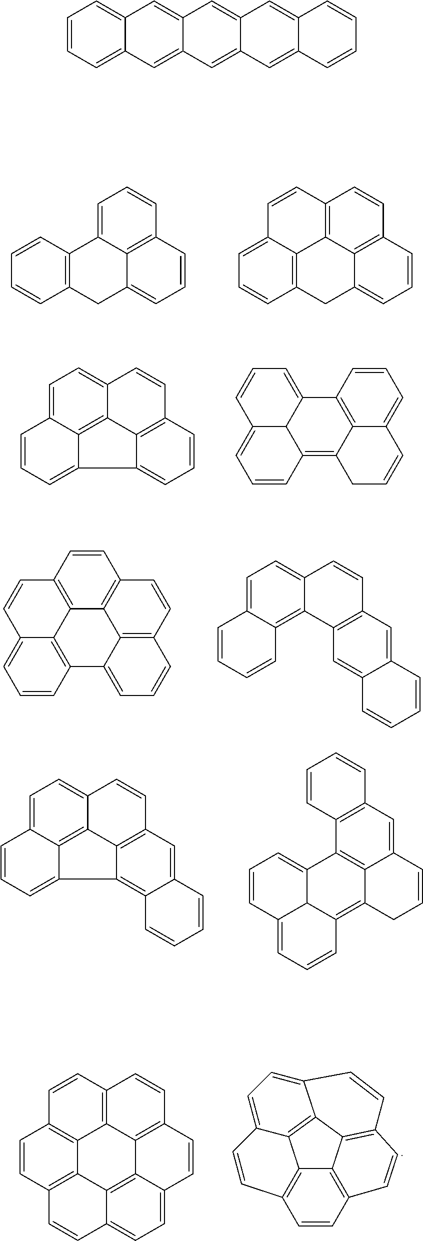

6. The hardmask composition as claimed in claim 1, wherein in Chemical Formula 1,
Ar¹ to Ar³ are each independently a substituted or unsubstituted group of a moiety of Group 1-2, and
Ar¹¹, Ar²¹, and Ar³¹ are each independently a substituted or unsubstituted group of a moiety of Group 2-1:

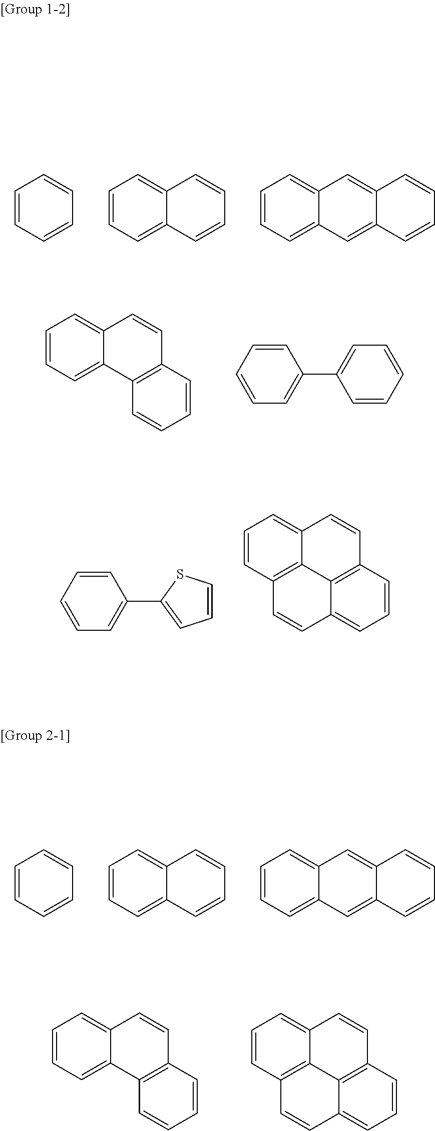

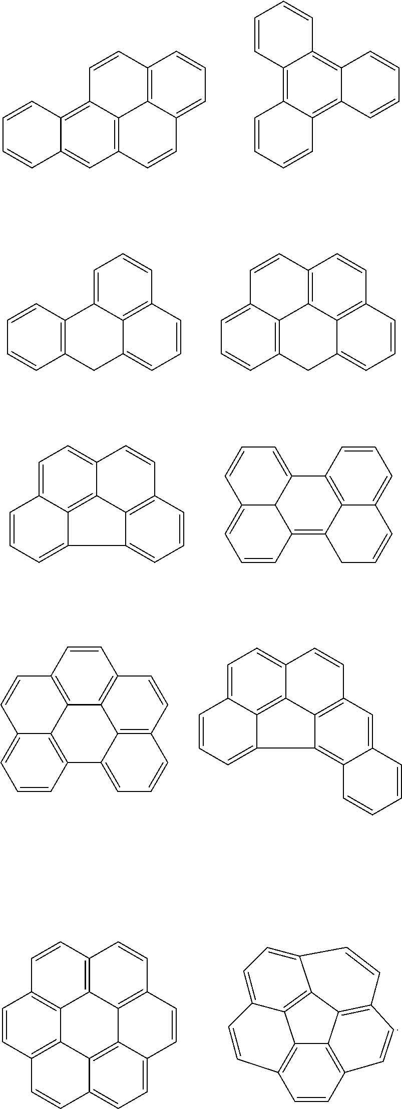

7. The hardmask composition as claimed in claim 1, wherein in Chemical Formula 1,
R¹ to R³ are each independently hydrogen, deuterium, a substituted or unsubstituted C1 to C30 saturated or unsaturated aliphatic hydrocarbon group, a substituted or unsubstituted C6 to C30 aromatic hydrocarbon group, or a combination thereof, and
R¹¹, R²¹, and R³¹ are each independently hydrogen, deuterium, or a substituted or unsubstituted C1 to C30 saturated or unsaturated aliphatic hydrocarbon group.

8. The hardmask composition as claimed in claim 1, wherein in Chemical Formula 1, R¹ to R³, R¹¹, R²¹, and R³¹ are each independently hydrogen, deuterium, a substituted or unsubstituted C1 to C20 alkyl group, a substituted or unsubstituted C6 to C20 aromatic hydrocarbon group, or a combination thereof.

9. The hardmask composition as claimed in claim 1, wherein in Chemical Formula 1, Ar¹ is the same as Ar², Ar¹¹ is the same as Ar²¹, R¹ is the same as R², or R¹¹ is the same as R²¹.

10. The hardmask composition as claimed in claim 1, wherein the compound represented by Chemical Formula 1 is represented by one of Chemical Formula 2 to Chemical Formula 15:

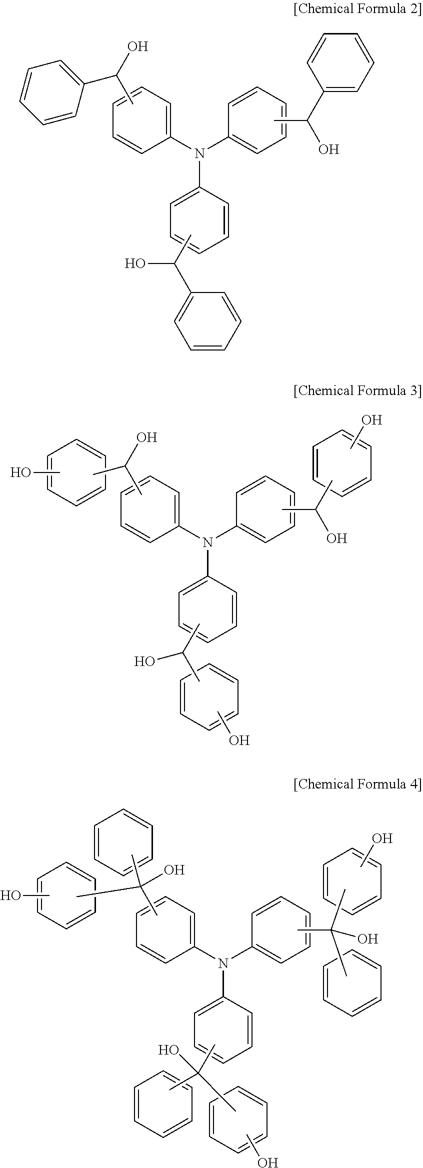

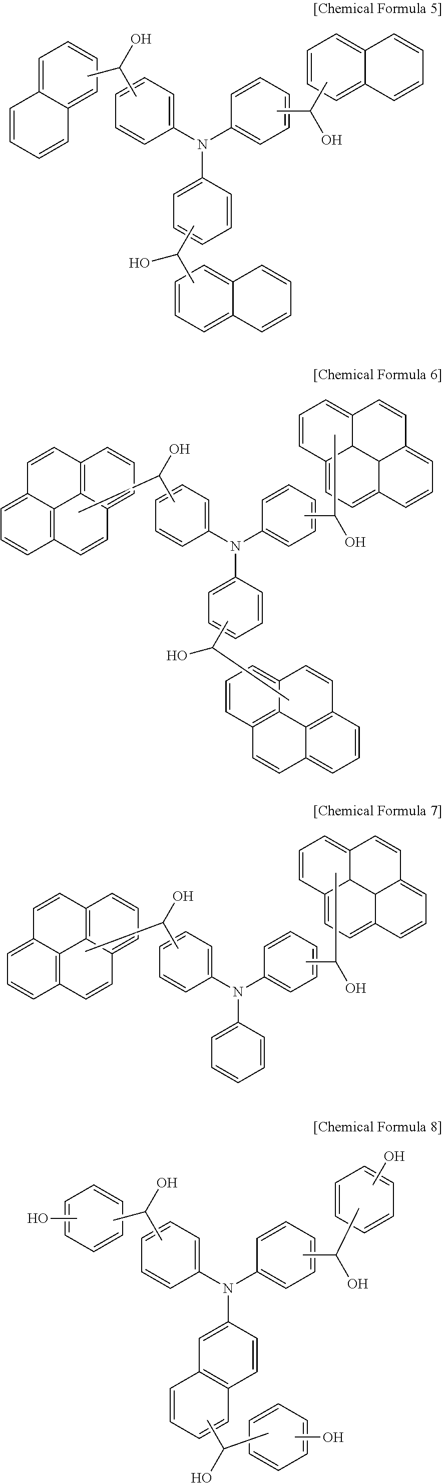

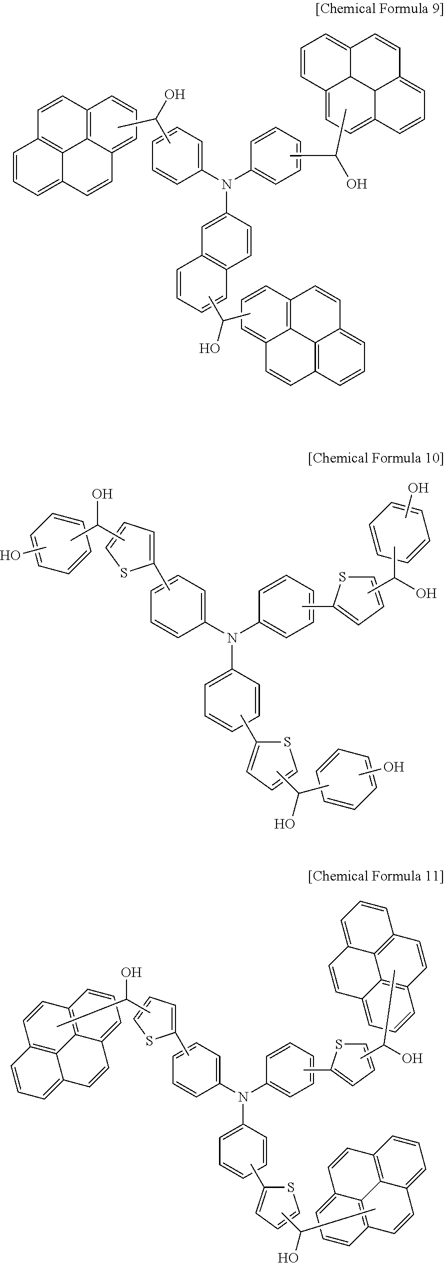

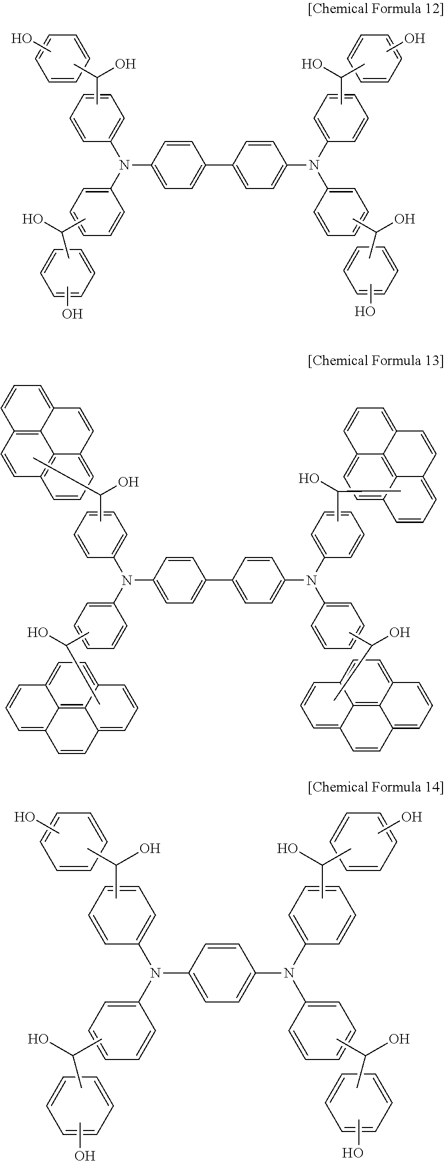

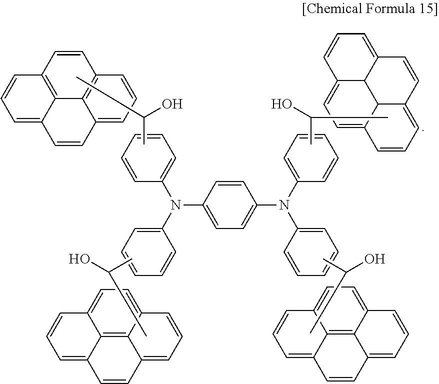

11. The hardmask composition as claimed in claim 1, wherein the compound has a molecular weight of about 500 g/mol to about 10,000 g/mol.

12. The hardmask composition as claimed in claim 1, wherein the compound is included in an amount of about 0.1 wt % to about 30 wt %, based on a total weight of the hardmask composition.

13. The hardmask composition as claimed in claim 1, wherein the solvent includes propylene glycol, propylene glycol diacetate, methoxy propanediol, diethylene glycol, diethylene glycol butylether, tri(ethylene glycol)monomethylether, propylene glycol monomethylether, propylene glycol monomethylether acetate, cyclohexanone, ethyl lactate, gamma-butyrolactone, N,N-dimethyl formamide, N,N-dimethyl acetamide, methylpyrrolidone, methylpyrrolidinone, acetylacetone, or ethyl 3-ethoxypropionate.

14. A hardmask layer comprising a cured product of the hardmask composition as claimed in claim 1.

15. A method of forming patterns, the method comprising:
providing a material layer on a substrate;
applying the hardmask composition as claimed in claim 1 to the material layer;
heat-treating the hardmask composition to form a hardmask layer;
forming a photoresist layer on the hardmask layer;
exposing and developing the photoresist layer to form a photoresist pattern;
selectively removing the hardmask layer using the photoresist pattern to expose a portion of the material layer; and
etching an exposed part of the material layer.

16. The method as claimed in claim 15, wherein forming the hardmask layer includes heat-treating at about 100° C. to about 1,000° C.
